(19) United States
(12) Patent Application Publication
(10) Pub. No.: US 2005/0181451 A1
(43) Pub. Date: Aug. 18, 2005

(54) Detection of asymptomatic coronary artery disease using atherogenic proteins and acute phase reactants

(76) Inventor: Harold M. Bates, East Brunswick, NJ (US)

Correspondence Address: Stephen P. Gilbert, Esq.;BRYAN CAVE LLP, 1290 Avenue of the Americas, New York, NY, 10104, US

(21) Appl. No.: 10/777,543

(22) Filed: Feb. 12, 2004

(51) Int. Cl. G01N 33/53; G06F 19/00; G01N 33/48; G01N 33/50

(52) U.S. Cl. 435/7.1; 702/19

(57) ABSTRACT

A method of assessing the likelihood that a person who is asymptomatic for coronary artery disease does in fact have the disease is disclosed. The levels of an atherogenic protein and acute phase reactant and optionally of anti-atherogenic protein for an individual are obtained and compared to one or more cut-points related to those substances and, based on the comparison(s), an assessment is made of the likelihood that the individual has coronary artery disease. The atherogenic protein may be OxLDL, the acute phase reactant may be C-reactive protein or fibrinogen, and the anti-atherogenic protein may be HDL.

### BACKGROUND OF THE INVENTION

The present invention relates to the field of coronary artery disease. More specifically, it relates to improving the detection of coronary artery disease in patients who are asymptomatic and particularly patients from the general population who are asymptomatic.

Coronary artery disease (“CAD”) is the leading cause of death in westernized countries; however, the problems of detecting coronary artery disease are well-known. Thus, more than half of the over 650,000 individuals who die each year in the United States from coronary artery disease are asymptomatic for the disease prior to their deaths (i.e., they had no recognized symptoms of coronary artery disease, such as chest pains).

U.S. Pat. No. 5,380,667 (issued in 1995) noted that most individuals with heart disease were largely asymptomatic until their first heart attack, that the major risk factors identified in the prior art were not perfect predictors (particularly for predicting the risk of coronary artery disease in any single individual), and that thirty to forty percent of the population was still misdiagnosed using the known major risk factors (column 1, lines 31-39). (All of the documents mentioned or otherwise referenced herein are incorporated herein in their entireties for all purposes.)

U.S. Pat. No. 5,756,067 (issued in 1998) noted that the tests then currently available to measure the risk of developing atherosclerosis included measuring the plasma content of cholesterol, triglycerides, and lipoproteins but that it was clear that these tests were not conclusive because approximately one-half of heart disease due to atherosclerosis occurred in patients with plasma triglycerides and cholesterol within the normal ranges of the population and because angiographic evidence of atherosclerosis had been found in patients with normal lipid levels.

Sasavage N, “Predicting Coronary Artery Disease, New Markers Could Identify Patients At Risk,” Clin. Lab. News March 1998, pages 6-7, noted that oxidation of low density lipoproteins might render it more atherogenic, that coronary artery disease appeared to be a multifactorial disease, and that those working in this area agreed that development of a new generation of biochemical markers would allow clinicians to better assess patient risk and intervene with treatments to avoid adverse outcomes.

The ability to determine with sufficient diagnostic accuracy whether an individual has or is likely in the future to have coronary artery disease has been a long-standing goal of medical science. Researchers have made many attempts to determine and establish sufficiently accurate markers for atherosclerosis and methods for predicting the likelihood that an individual will in the future have coronary artery disease. For example, attempts have been made to provide monoclonal antibodies that recognize various low density lipoprotein (“LDL”) substances and/or other substances that might be associated with atherosclerosis and/or thrombosis. See, e.g., the following documents, which concern the foregoing as well as coronary artery disease, lipids, and assays in general: U.S. Pat. Nos. 5,024,829, 5,026,537, 5,046,499, 5,120,834, 5,196,324, 5,223,410, 5,362,649, 5,380,667, 5,396,886, 5,453,359, 5,487,892, 5,597,726, 5,604,105, 5,658,729, 5,690,103, 5,710,008, 5,731,208, 5,756,067, 6,040,147, and 6,309,888; U.S. Patent Application Nos. 2003/0100486 and 2003/0152566; non-U.S. patent documents EP 0 327 418 A1, EP 0 433 088 B1, EP 0 484 863 A1, WO 94/23302, WO 98/59248, WO 00/14548, JP Laid Open Patent Application (Kokai) No. 8-304395, and JP Laid Open Patent Application (Kokai) No. 9-5323; Adams et al., “Cardiac Troponin I, A Marker With High Specificity For Cardiac Injury,” Circulation 1993; 88(1): 101-106; American Biogenetic Sciences Inc., 1995 Annual Report, 24 pages (1995); American Biogenetic Sciences, Focus on Diagnostic Tests: A Technology Analysis. Updated Full Report, 33 pages, Paisley and Habermas, Inc. (Jun. 3, 1996); American Biogenetic Sciences, Inc., “Renal dialysis joint venture announced by American Biogenetic Sciences, Inc. and Gull Laboratories, Inc.,” News Release (Sep. 26, 1996); American Biogenetic Sciences, Inc., Jesup & Lamont Securities Corporation, “New Buy Recommendation dated Mar. 28, 1996” (12 pages); Antman et al., “Cardiac-Specific Troponin I Levels To Predict The Risk Of Mortality In Patients With Acute Coronary Syndromes,” N. Eng. J. Med. 1996; 335(18): 1342-1349; AtheroGenics, Inc. Printout of Web Site (WWW.ATHEROGENICS.COM), Home page and “Technology Platform” and “In The News” sections, 17 pages (printed Jun. 8, 1998); Aviram et al., “Phospholipase D-Modified Low Density Lipoprotein Is Taken Up By Macrophages At Increased Rate, A Possible Role For Phosphatidic Acid.” J. Clin. Invest. 1993; 91: 1942-1952; Berliner et al., “The Role Of Oxidized Lipoproteins In Atherogenesis,” Free Radical Biology & Medicine 1996; 20(5): 707-727; Boyd et al., “Direct Evidence For A Protein Recognized By A Monoclonal Antibody Against Oxidatively Modified LDL In Atherosclerotic Lesions From A Watanabe Hyperlipidemic Rabbit,” Am. J. Pathol. 1989 November; 135(5): 815-825; Brody, “Hunt For Heart Disease Tracks A New Suspect,” The New York Times, 3 pages (Jan. 6, 2004); Brown et al., “Lipoprotein Metabolism In The Macrophage: Implications For Cholesterol Deposition In Atherosclerosis,” Annu. Review Biochem. 1983; 52: 223-261; Cartier et al., “Chronic Exposure To Cyclosporin Affects Endothelial And Smooth Muscle Reactivity In The Rat Aorta,” Ann. Thorac. Surg. 1994; 58: 789-794; Chapelle, “How Should We proceed When A Myocardial Infarction Is Suspected,” Acta Clinica Belgica 1984; 39(6): 393-395; Chen et al., “Basic Fibroblast Growth Factor Reverses Atherosclerotic Impairment Of Human Coronary Angiogenesis-Like Responses In Vitro,” Atherosclerosis 1995; 116: 261-268; Chin et al., “Inactivation Of Endothelial Derived Relaxing Factor By Oxidized Lipoproteins,” J. Clin. Invest. 1992; 89: 10-18; Cockcroft et al., “Prediction of creatinine clearance from serum creatinine,” Nephron 1976; 16: 31-41; Crisp et al., “Antiendothelial Antibodies After Heart Transplantation: The Accelerating Factor In Transplant-Associated Coronary Artery Disease?” J. Heart Lung Transplant. 1994; 13(1, Part 1): 81-92; Declerck et al., “Fibrinolytic Response And Fibrin Fragment D-Dimer Levels In Patients With Deep Vein Thrombosis,” Thromb. Haemost. 1987; 58(4): 1024-1029; Degoulet et al., “Mortality Risk Factors In Patients Treated By Chronic Hemodialysis,” Nephron 1982; 31: 103-110; Esterbauer et al., “Autooxidation Of Human Low Density Lipoprotein: Loss Of Polyunsaturated Fatty Acids And Vitamin E And Generation Of Aldehydes,” J. Lipid Res. 1987; 28: 495-509; Farber et al., “Differences In Prostaglandin Metabolism In Cultured Aortic And Pulmonary Arterial Endothelial Cells Exposed To Acute And Chronic Hypoxia,” Circ. Res. 1991; 68(5): 1446-1457; Fogelman et al., “Malondialdehyde Alteration Of Low Density Lipoproteins Leads To Cholesteryl Ester Accumulation In Human Monocyte-Macrophages,” Proc. Natl. Acad. Sci. USA 1980; 77(4): 2214-2218; Folcik et al., “Lipoxygenase Contributes To The Oxidation Of Lipids In Human Atherosclerotic Plaques,” J. Clin. Invest. 1995; 96: 504-510; Friedman et al., “Hyperhomocysteinemia As A Risk Factor For Cardiovascular Disease In Patients Undergoing Hemodialysis,” Nutr. Rev. 1995; 53(7): 197-201; Galle et al., “Oxidized Lipoprotein(A) Inhibits Endothelium-Dependent Dilation: Prevention By High Density Lipoprotein,” Eur. J. Pharmacol. 1994; 265: 111-115; Galle et al., “Cyclosporin And Oxidized Low Density Lipoproteins Synergistically Potentiate Vasoconstriction: Influence Of The Endothelium,” Eur. Heart J. 1993; 14(Suppl. I): 111-117; Gerrity, “The Role Of The Monocyte In Atherogenesis. I. Transition Of Blood-Borne Monocytes Into Foam Cells In Fatty Lesions,” Am. J. Pathol. 1981; 103(2): 181-190; Grattan et al., “Cytomegalovirus Infection Is Associated With Cardiac Allograft Rejection And Atherosclerosis,” J. Am. Med. Assoc. 1989; 261(24): 3561-3566; Haberland et al., “Specificity Of Receptor-Mediated Recognition Of Malondialdehyde-Modified Low Density Lipoproteins,” Proc. Natl. Acad. Sci USA. 1982; 79: 1712-1716; Haberland et al., “Role Of Lysines In Mediating Interaction Of Modified Low Density Lipoproteins With The Scavenger Receptor Of Human Monocyte Macrophages,” J. Biol. Chem. 1984; 259(18): 11305-11311; Hamm et al., “Emergency Room Triage Of Patients With Acute Chest Pain By Means Of Rapid Testing For Cardiac Troponin T Or Troponin I,” N. Eng. J. Med. 1997; 337(23): 1648-1653; Hamm et al., “Emergency Room Triage Of Patients With Acute Chest Pain By Means Of Rapid Testing For Cardiac Troponin T Or Troponin I,” N. Eng. J. Med. 1997; 337(23): 1648-1653. Letters concerning same and authors' reply, published in N. Eng. J. Med. 1998; 338(18): 1314-1315; Hammer et al., “Generation, Characterization, And Histochemical Application Of Monoclonal Antibodies Selectively Recognizing Oxidatively Modified ApoB-Containing Serum Lipoproteins,” Arterioscler. Thromb. Vasc. Biol. 1995; 15(5): 704-713; Hansson et al. (eds.). Immune Functions of the Vessel Wall, Volume II (Harwood Academic Publishers 1996). Chapter 9: Witztum J L, Palinski W. “Autoimmunity To Oxidized Lipoproteins,” Pages 159-171; Havel et al., “The Distribution And Chemical Composition Of Ultracentrifugally Separated Lipoproteins In Human Serum,” J. Clin. Invest. 1955; 34: 1345-1353; Heery et al., “Oxidatively Modified LDL Contains Phospholipids With Platelet-Activating Factor-Like Activity And Stimulates The Growth Of Smooth Muscle Cells,” J. Clin. Invest. 1995; 96: 2322-2330; Hirschfield et al., “C-reactive protein and cardiovascular disease: new insights from an old molecule,” Q J. Med. 2003 November; 96(11): 793-807; Hlatky, “Evaluation Of Chest Pain In The Emergency Department,” N. Eng. J. Med. 1997; 337(23): 1687-1689; Hoff et al., “Lesion-Derived Low Density Lipoprotein And Oxidized Low Density Lipoprotein Share A Lability For Aggregation, Leading To Enhanced Macrophage Degradation,” Arterioscler. Thromb. 1991; 11(5): 1209-1222; Hoff et al., “Modification Of Low Density Lipoprotein With 4-Hydroxynonenal Induces Uptake By Macrophages,” Arteriosclerosis 1989; 9(4): 538-549; Hoffmeister et al., “Alterations Of Coagulation And Fibrinolytic And Kallikrein-Kinin Systems In The Acute And Post-Acute Phases In Patients With Unstable Angina Pectoris,” Circulation 1995; 91(10): 2520-2527; Holvoet et al., “Stimulation With A Monoclonal Antibody (mAb4E4) Of Scavenger Receptor-Mediated Uptake Of Chemically Modified Low Density Lipoproteins By THP-1-Derived Macrophages Enhances Foam Cell Generation,” J. Clin. Invest. 1994; 93: 89-98; Holvoet et al., “Beta-VLDL Hypercholesterolemia Relative To LDL Hypercholesterolemia Is Associated With Higher Levels Of Oxidized Lipoproteins And A More Rapid Progression Of Coronary Atherosclerosis In Rabbits,” Arterioscler. Thromb. Vasc. Biol. 1997; 17(11): 2376-2382; Holvoet et al., “Oxidized Lipoproteins In Atherosclerosis And Thrombosis,” FASEB J. 1994; 8: 1279-1284; Holvoet et al., “Thrombosis And Atherosclerosis,” Curr. Opinion Lipidol. 1997; 8: 320-328; Holvoet et al., “Malondialdehyde-Modified Low Density Lipoproteins In Patients With Atherosclerotic Disease,” J. Clin. Invest. 1995; 95: 2611-2619; Holvoet et al., “Correlation Between Oxidized Low Density Lipoproteins And Von Willebrand Factor In Chronic Renal Failure,” Thromb. Haemost. 1996; 76(5): 663-669; Holvoet et al., “Correlation Between Oxidized Low Density Lipoproteins And Coronary Artery Disease In Heart Transplant Patients,” Abstract published in Final Programme of 66th Congress of the European Atherosclerosis Society, Florence (Italy), Jul. 13-14, 1996; Abstract Book, page 47; Holvoet et al., “Oxidized Low Density Lipoproteins In Patients With Transplant-Associated Coronary Artery Disease,” Arterioscler. Thromb. Vasc. Biol. 1998; 18(1): 100-107; Holvoet et al., “LDL Hypercholesterolemia Is Associated With Accumulation Of Oxidized LDL, Atherosclerotic Plaque Growth, And Compensatory Vessel Enlargement In Coronary Arteries Of Miniature Pigs,” Arterioscler. Thromb. Vasc. Biol. 1998; 18: 415-422; Holvoet et al., Presentation at 70th Scientific Session Of The American Heart Association, Orlando, Fla., November 9-12, and published in abstract form in Circulation 1997; 96(Suppl. I): 1417 (Abstract 2328); Holvoet et al., “Oxidized LDL And Malondialdehyde-Modified LDL In Patients With Acute Coronary Syndromes And Stable Coronary Artery Disease,” Circulation 1998; 98: 1487-1494; Holvoet et al., “Malondialdehyde-Modified LDL As A Marker Of Acute Coronary Syndromes,” J. Am. Med. Assoc. 1999; 281(18): 1718-1721; Holvoet P, “Oxidative Modification Of Low-Density Lipoproteins In Atherothrombosis,” Acta Cardiol. 1998; 53(5): 253-260; Holvoet et al., “Circulating oxidized LDL is a useful marker for identifying patients with coronary artery disease,” Arterioscler Thromb Vasc Biol. 2001 May; 21(5):844-8; Holvoet P et al., “Association of high coronary heart disease risk status with circulating oxidized LDL in the well-functioning elderly: findings from the Health, Aging, and Body Composition study,” Arterioscler Thromb Vasc Biol. 2003 August; 23(8): 1444-1448; Hruban et al., “Accelerated Arteriosclerosis In Heart Transplant Recipients Is Associated With A T-Lymphocyte-Mediated Endothelialitis,” Am. J. Pathol. 1990; 137(4): 871-882; Hulthe J et al., “Circulating oxidized LDL is associated with subclinical atherosclerosis development and inflammatory cytokines (AIR Study),” Arterioscler Thromb Vasc Biol. 2002 Jul. 1; 22(7): 1162-1167; Itabe et al., “A Monoclonal Antibody Against Oxidized Lipoprotein Recognizes Foam Cells In Atherosclerotic Lesions: Complex Formation Of Oxidized Phosphatidylcholines And Polypeptides,” J. Biol. Chem. 1994; 269(21): 15274-15279; Itabe et al., “Sensitive Detection Of Oxidatively Modified Low Density Lipoprotein Using A Monoclonal Antibody,” J. Lipid Res. 1996; 37: 45-53; Juckett et al., “Ferritin Protects Endothelial Cells From Oxidized Low Density Lipoprotein In Vitro,” Am. J. Pathol. 1995; 147(3): 782-789; Kaplan et al., “Renal Vasoconstriction Caused By Short-Term Cholesterol Feeding Is Corrected By Thromboxane Antagonist Or Probucol,” J. Clin. Invest. 1990; 86: 1707-1714; Keane et al., “Hyperlipidemia And Progressive Renal Disease,” Kidney Int. 1991; 39(Suppl.): S41-S48; Kolata, “A New Generation Of Tests To Determine Heart Trouble,” New York Times News Service, 7 pages (Nov. 26, 1995); Koskinen et al., “Acute Cytomegalovirus Infection Induces A Subendothelial Inflammation (Endothelialitis) In The Allograft Vascular Wall, A Possible Linkage With Enhanced Allograft Arteriosclerosis,” Am. J. Pathol. 1994; 144(1): 41-50; Kotani et al., “Distribution Of Immunoreactive Malondialdehyde-Modified Low-Density Lipoprotein In Human Serum,” Biochimica et Biophysica Acta 1994; 1215: 121-125; Lee et al., “Serum Enzymes In The Diagnosis Of Acute Myocardial Infarction, Annals of Internal Medicine 1986; 105: 221-223; Libby et al., “Functions Of Vascular Wall Cells Related To Development Of Transplantation-Associated Coronary Arteriosclerosis,” Transplant. Proc. 1989; 21(4): 3677-3684; Lynch et al., “Formation Of Non-Cyclooxygenase-Derived Prostanoids (F₂-Isoprostanes) In Plasma And Low Density Lipoprotein Exposed To Oxidative Stress In Vitro,” J. Clin. Invest. 1994; 93: 998-1004; Mabile et al., “Alpha-Tocopherol And Trolox Block The Early Intracellular Events (TBARS And Calcium Rises) Elicited By Oxidized Low Density Lipoproteins In Cultured Endothelial Cells,” Free Radic. Biol. Med. 1995; 19(2): 177-187; Major et al., “Increased Cholesterol Efflux In Apolipoprotein AI (ApoAI)-Producing Macrophages As A Mechanism For Reduced Atherosclerosis In ApoAI((−/−)) mice,” Arterioscler Thromb Vasc Biol. 2001 November; 21(11): 1790-1795; Menschikowski et al., “Secretory Group II Phospholipase A2 In Human Atherosclerotic Plaques,” Atherosclerosis 1995; 118: 173-181; McCully, “Chemical Pathology Of Homocysteine. I. Atherogenesis.” Ann. Clin. Lab. Sci. 1993; 23(6): 477-493; Morrow et al., “Non-Cycloogenase-Derived Prostanoids (F₂-isoprostanes) Are Formed In Situ On Phospholipids,” Proc. Natl. Acad. Sci. USA 1992; 89: 10721-10725; Muldoon et al., Ryan et al., Oltrona et al., and Liuzzo et al., Letters and reply by authors, “C-Reactive Protein And Serum Amyloid A Protein In Unstable Angina,” N. Engl. J. Med. 1995; 332(6): 398-400; Murugesan et al., “Oxidized Low Density Lipoprotein Inhibits The Migration Of Aortic Endothelial Cells In Vitro,” J. Cell. Biol. 1993; 120(4): 1011-1019; Neff et al., “Patients Surviving 10 Years Of Hemodialysis,” Am. J. Med. 1983; 74: 996-1004; Ohman et al., “Cardiac Troponin T Levels For Risk Stratification In Acute Myocardial Ischemia,” N. Eng. J. Med. 1996 335(18): 1333-1341; O'Marcaigh et al., “Estimating The Predictive Value Of A Diagnostic Test, How To Prevent Misleading Or Confusing Results,” Clin. Ped. 1993; 32(8): 485-491; Palinski et al., “Low Density Lipoprotein Undergoes Oxidative Modification In Vivo,” Proc. Natl. Acad. Sci. USA 1989; 86: 1372-1376; Palinski et al., “Antisera And Monoclonal Antibodies Specific For Epitopes Generated During Oxidative Modification Of Low Density Lipoprotein,” Arteriosclerosis 1990; 10(3): 325-335; Parthasarathy et al., “A Role For Endothelial Cell Lipoxygenase In The Oxidative Modification Of Low Density Lipoprotein,” Proc. Nat. Acad. Sci. USA 1989; 86: 1046-1050; Penn et al., “Oxidized lipoproteins, altered cell function and atherosclerosis,” Atherosclerosis 1994; 108(Suppl.): S21-S29; Pocock, Clinical Trials. A Practical Approach, Chapter 14: “Further Aspects Of Data Analysis,” Pages 211-233, John Wiley & Sons. 1993; Rasmussen et al., “Decrease Of Von Willebrand Factor Levels After A High-Monounsaturated Fat Diet In Non-Insulin-Dependent Diabetic Subjects,” Metabolism 1994; 43(11): 1406-1409; Ravalli et al., “Immunohistochemical Demonstration Of 15-Lipoxygenase In Transplant Coronary Artery Disease,” Arterioscler. Thromb. Vasc. Biol. 1995; 15(3): 340-348; Reade et al., “Expression Of Apolipoprotein B Epitopes In Low Density Lipoproteins Of Hemodialyzed Patients,” Kidney Int. 1993; 44: 1360-1365; Reverter et al., “Platelet Activation During Hemodialysis Measured Through Exposure Of P-Selectin: Analysis By Flow Cytometric And Ultrastructural Techniques,” J. Lab. Clin. Med. 1994; 124(1): 79-85; Ridker et al., “C-Reactive Protein Adds To The Predictive Value Of Total And HDL Cholesterol In Determining Risk Of First Myocardial Infarction,” Circulation 1998; 97:2007-2011; Ridker et al., “Prospective study of C-reactive protein and the risk of future cardiovascular events in stable and unstable angina,” Circulation 1998; 98:731-733; Rose et al., “Humoral immune responses after cardiac transplantation: correlation with fatal rejection and graft atherosclerosis,” Surgery 1989; 106(2): 203-208; Rosenfeld et al., “Distribution Of Oxidation Specific Lipid-Protein Adducts And Apolipoprotein B In Atherosclerotic Lesions Of Varying Severity From WHHL Rabbits,” Arteriosclerosis 1990; 10(3): 336-349; Ross, “The Pathogenesis Of Atherosclerosis: A Perspective For The 1990s,” Nature 1993; 362: 801-809; Salonen et al., “Autoantibody Against Oxidised LDL And Progression Of Carotid Atherosclerosis,” Lancet 1992; 339(8798): 883-887; Sasavage, “Predicting Coronary Artery Disease, New Markers Could Identify Patients At Risk,” Clin. Lab. News March 1998; pages 6-7; Savenkova et al., “Tyrosyl Radical Generated By Myeloperoxidase Is A Physiological Catalyst For The Initiation Of Lipid Peroxidation In Low Density Lipoprotein,” J. Biol. Chem. 1994; 269(32): 20394-20400; Schaffner et al., “Arterial Foam Cells With Distinctive Immunomorphologic And Histochemical Features Of Macrophages,” Am. J. Pathol. 1980; 100(1): 57-80; Schonbeck et al., “Oxidized Low-Density Lipoprotein Augments And 3-Hydroxy-3-Methylglutaryl Coenzyme A Reductase Inhibitors Limit CD40 And CD401 Expression In Human Vascular Cells,” Circulation 2002; 106(23): 2888-2893; Shacter, “Quantification And Significance Of Protein Oxidation In Biological Samples,” Drug Metab Rev. 2000 August-November; 32(3-4): 307-26; Shultz, “Clinical Interpretation Of Laboratory Procedures,” Chapter 14 in Teitz, Fundamentals of Clinical Chemistry, Burtis et al. (eds.), 4th edition 1996, W.B.Saunders Company, Pages 192-199; Schulz et al., “Preserved Antioxidative Defense Of Lipoproteins In Renal Failure And During Hemodialysis,” Am. J. Kidney Dis. 1995; 25(4): 564-571; Selwyn et al., “Atherogenic Lipids, Vascular Dysfunction, And Clinical Signs Of Ischemic Heart Disease,” Circulation 1997; 95(1): 5-7; Sparrow et al., “Cellular Oxidative Modification Of Low Density Lipoprotein Does Not Require Lipoxygenases,” Proc. Nat. Acad. Sci. USA 1992; 89: 128-131; Sparrow et al., “Enzymatic Modification Of Low Density Lipoprotein By Purified Lipoxygenase Plus Phospholipase-A₂ Mimic Cell-Mediated Oxidative Modification,” J. Lipid Res. 1988; 29: 745-753; Steinberg et al., “Lipoproteins And Atherogenesis: Current Concepts,” J. Am. Med. Assoc. 1990; 264(23): 3047-3052; Steinberg, “Clinical Trials Of Antioxidants In Atherosclerosis: Are We Doing The Right Thing?” Lancet 1995; 346: 36-38; Steinberg, “Lewis A. Conner Memorial Lecture, Oxidative Modification Of LDL And Atherogenesis,” Circulation 1997; 95: 1062-1071; Steinbrecher et al., “Modification Of Low Density Lipoprotein By Endothelial Cells Involves Lipid Peroxidation And Degradation Of Low Density Lipoprotein Phospholipids,” Proc. Nat. Acad. Sci. USA 1984; 81: 3883-3887; Steinbrecher, “Oxidation Of Low Density Lipoprotein Results In Derivatization Of Lysine Residues Of Apolipoprotein B By Lipid Peroxide Decomposition Products,” J. Biol. Chem. 1987; 262(8): 3603-3608; Steinbrecher et al., “Scavenger Receptor-Independent Stimulation Of Cholesterol Esterification In Macrophages By Low Density Lipoprotein Extracted From Human Aortic Intima,” Arterioscler. Thromb. 1992; 12(5): 608-625; Sutherland et al., “Oxidation Of Low Density Lipoproteins From Patients With Renal Failure Or Renal Transplants,” Kidney Int. 1995; 48: 227-236; Tamai et al., “Single LDL Apheresis Improves Endothelium-Dependent Vasodilation In Hypercholesterolemic Humans,” Circulation 1997; 95(1): 76-82; Tanaka et al., “Endothelial And Smooth Muscle Cells Express Leukocyte Adhesion Molecules Heterogeneously During Acute Rejection Of Rabbit Cardiac Allografts,” Am. J. Pathol. 1994; 144(5): 938-951; Trachtman et al., “Dietary Vitamin E Supplementation Ameliorates Renal Injury In Chronic Puromycin Aminonucleoside Nephropathy,” J. Am. Soc. Nephrol. 1995; 5(10): 1811-1819; Tuzcu et al., “Occult And Frequent Transmission Of Atherosclerotic Coronary Disease With Cardiac Transplantation. Insights From Intravascular Ultrasound,” Circulation 1995; 91(6): 1706-1713; Uchida K et al., “Protein-Bound Acrolein: Potential Markers For Oxidative Stress,” Proc. Natl. Acad. Sci. USA 1998; 95: 4882-4887; Van de Werf, “Cardiac Troponins In Acute Coronary Syndromes,” N. Eng. J. Med. 1996; 335(18): 1388-1389; Varo et al., “Soluble CD40L: Risk Prediction After Acute Coronary Syndromes,” Circulation 2003; 108(9): 1049-1052; Wentworth et al., “Evidence for ozone formation in human atherosclerotic arteries,” Science 2003; 302(5647): 1053-1056; Yla-Herttuala et al., “Evidence For The Presence Of Oxidatively Modified Low Density Lipoprotein In Atherosclerotic Lesions Of Rabbit And Man,” J. Clin. Invest. 1989 October; 84: 1086-1095; Zaidi et al., “A Rapid Method For Preparation Of Sarcolemma From Frog Leg Skeletal Muscle,” Chemical Abstracts 1982 Jun. 7; 96(23): 196091e; Zawadzki et al., “An Immunochemical Marker Of Low Density Lipoprotein Oxidation,” J. Lipid Res. 1989; 30: 885-891; Zhao et al., “Oxidized LDL Induces Serotonin Release From Blood Platelets,” Am. J. Hematol. 1995; 48: 285-287; Zwaginga et al., “Thrombus Formation And Platelet-Vessel Wall Interaction In The Nephrotic Syndrome Under Flow Conditions,” J. Clin. Invest. 1994; 93: 204-211; Zweig et al., “ROC Curve Analysis: An Example Showing The Relationships Among Serum Lipid And Apolipoprotein Concentrations In Identifying Patients With Coronory Artery Disease,” Clin. Chem. 1992; 38(8): 1425-1428.

U.S. Pat. No. 6,040,147 (issued in 2000) suggests using systemic inflammatory markers (e.g., C-reactive protein) for characterizing an individual's risk profile of developing a future cardiovascular disorder. C-reactive protein is used alone or in combination with total cholesterol or the total cholesterol:HDL ratio.

In U.S. Pat. No. 6,309,888 (issued in 2001), Holvoet et al. have provided a method having a clinically sufficient degree of diagnostic accuracy for detecting the presence of coronary artery disease in a human patient from the general population and for distinguishing between the stages of the disease. The stages are, first, the non-acute stage, which is either asymptomatic coronary artery disease or stable angina, second, the acute stage known as unstable angina, and, third, the acute stage known as acute myocardial infarction. The diseased state (as opposed to the non-diseased state) is indicated by the clinically significant presence of a first marker in a sample from the patient. The presence of one of the two acute stages, unstable angina or acute myocardial infarction, is indicated by the clinically significant presence of a second marker in a sample from the patient. The presence of the more severe acute stage known as acute myocardial infarction is indicated by the clinically significant presence of a third marker in a sample from the patient. Preferably the first marker comprises OxLDL (oxidized low density lipoprotein), the second marker comprises MDA-modified LDL (malondialdehyde-modified low density lipoprotein), and the third marker is a troponin. Preferably the OxLDL and MDA-modified LDL are detected using monoclonal antibodies that can detect the presence of those markers in undiluted human plasma at concentrations as low as 0.02 milligrams/deciliter. Substances discussed in the examples include OxLDL, MDA-modified LDL, HDL, and C-reactive protein. The antibodies that can be used have high affinity and bind MDA-modified LDL and/or OxLDL whose apo B-100 moieties contain at least 60 substituted lysine residues per apo B-100 moiety. The preferred monoclonal antibodies are mAb-4E6, mAb-1H11, and mAb-8A2.

Notwithstanding all of the foregoing, the need still remains for a method for detecting coronary artery disease in asymptomatic patients with as high a degree of accuracy as is possible.

### SUMMARY OF THE INVENTION

An invention satisfying that need and having advantages and benefits that will be apparent to one skilled in the art has now been developed. Broadly, this invention provides a method of making an assessment of the likelihood that a human patient who is asymptomatic for coronary artery disease has the disease, the method comprising the steps:
(a) obtaining the level of an atherogenic protein in a sample from the patient, obtaining the level of an acute phase reactant in a sample from the patient, and optionally obtaining the level of an anti-atherogenic protein in a sample from the patient;
(b) obtaining at least one of:
   (i) a first cut-point related to the atherogenic protein and a second cut-point related to the acute phase reactant,
   (ii) a third cut-point related to the atherogenic protein and the acute phase reactant,
   (iii) a fourth cut-point related to the atherogenic protein and the acute phase reactant and a fifth cut-point related to the anti-atherogenic protein,
   (iv) a sixth cut-point related to the atherogenic protein and a seventh cut-point related to the acute phase reactant and the anti-atherogenic protein,
   (v) an eighth cut-point related to the atherogenic protein and the anti-atherogenic protein and a ninth cut-point related to the acute phase reactant,
   (vi) a tenth cut-point related to the atherogenic protein, the acute phase reactant, and the anti-atherogenic protein,
   (vii) an eleventh cut-point related to the atherogenic protein, a twelfth cut-point related to the acute phase reactant, and a thirteenth cut-point related to the anti-atherogenic protein; and
(c) assessing whether the patient is likely to have asymptomatic coronary artery disease based on at least one of the following:
   (i) a comparison to the first cut-point of a first value related to the level of the atherogenic protein and a comparison to the second cut-point of a second value related to the level of the acute phase reactant,
   (ii) a comparison to the third cut-point of a third value related to the levels of the atherogenic protein and acute phase reactant,
   (iii) a comparison to the fourth cut-point of a fourth value related to the levels of the atherogenic protein and acute phase reactant and a comparison to the fifth cut-point of a fifth value related to the level of the anti-atherogenic protein,
   (iv) a comparison to the sixth cut-point of a sixth value related to the level of the atherogenic protein and a comparison to the seventh cut-point of a seventh value related to the levels of the acute phase reactant and anti-atherogenic protein,
   (v) a comparison to the eighth cut-point of an eighth value related to the levels of the atherogenic protein and anti-atherogenic protein and a comparison to the ninth cut-point of a ninth value related to the level of the acute phase reactant,
   (vi) a comparison to the tenth cut-point of a tenth value related to the levels of the atherogenic protein, acute phase reactant, and anti-atherogenic protein, and
   (vii) a comparison to the eleventh cut-point of an eleventh value related to the level of the atherogenic protein, a comparison to the twelfth cut-point of a twelfth value related to the level of the acute phase reactant, and a comparison to the thirteenth cut-point of a thirteenth value related to the level of the anti-atherogenic protein.

In another aspect, this invention provides a method of facilitating the assessment by a medical professional of the likelihood that a human patient who is asymptomatic for coronary artery disease has the disease, the method comprising the steps:
(a) obtaining the level of an atherogenic protein in a sample from the patient, obtaining the level of an acute phase reactant in a sample from the patient, and optionally obtaining the level of an anti-atherogenic protein in a sample from the patient;
(b) obtaining at least one of:
   (i) a first cut-point related to the atherogenic protein and a second cut-point related to the acute phase reactant,
   (ii) a third cut-point related to the atherogenic protein and the acute phase reactant,
   (iii) a fourth cut-point related to the atherogenic protein and the acute phase reactant and a fifth cut-point related to the anti-atherogenic protein,
   (iv) a sixth cut-point related to the atherogenic protein and a seventh cut-point related to the acute phase reactant and the anti-atherogenic protein,
   (v) an eighth cut-point related to the atherogenic protein and the anti-atherogenic protein and a ninth cut-point related to the acute phase reactant,
   (vi) a tenth cut-point related to the atherogenic protein, the acute phase reactant, and the anti-atherogenic protein, and
   (vii) an eleventh cut-point related to the atherogenic protein, a twelfth cut-point related to the acute phase reactant, and a thirteenth cut-point related to the anti-atherogenic protein;
(c) providing to the medical professional at least one of:
   (i) a first value related to the level of the atherogenic protein and a second value related to the level of the acute phase reactant,
   (ii) a third value related to the levels of the atherogenic protein and acute phase reactant,
   (iii) a fourth value related to the levels of the atherogenic protein and acute phase reactant and a fifth value related to the level of the anti-atherogenic protein,
   (iv) a sixth value related to the level of the atherogenic protein and a seventh value related to the levels of the acute phase reactant and anti-atherogenic protein,
   (v) an eighth value related to the levels of the atherogenic protein and anti-atherogenic protein and a ninth value related to the level of the acute phase reactant, and
   (vi) a tenth value related to the levels of the atherogenic protein, acute phase reactant, and anti-atherogenic protein,
   (vii) an eleventh value related to the level of the atherogenic protein, a twelfth value related to the level of the acute phase reactant, and a thirteenth value related to the level of the anti-atherogenic protein; and
(d) providing to the medical professional the appropriate one or more of the cut-points to permit the medical professional to assess whether the patient is likely to have asymptomatic coronary artery disease based on at least one of the following:
   (i) a comparison to the first cut-point of the first value and a comparison to the second cut-point of the second value,
   (ii) a comparison to the third cut-point of the third value,
   (iii) a comparison to the fourth cut-point of the fourth value and a comparison to the fifth cut-point of the fifth value,
   (iv) a comparison to the sixth cut-point of the sixth value and a comparison to the seventh cut-point of the seventh value,
   (v) a comparison to the eighth cut-point of the eighth value and a comparison to the ninth cut-point of the ninth value,
   (vi) a comparison to the tenth cut-point of the tenth value, and
   (vii) a comparison to the eleventh cut-point of the eleventh value, a comparison to the twelfth cut-point of the twelfth value, and a comparison to the thirteenth cut-point of the thirteenth value.

Unless otherwise explicitly or implicitly required by the claims themselves, the steps of a claim may be performed in any sequence and at any time and with no maximum time between steps. For example, a clinical chemistry laboratory would still be performing a process of this invention if it provided to a medical professional values related to the substances of interest for a particular patient even if it had provided the appropriate cut-point(s) to the medical professional several months before it provided the values for the patient.

In some embodiments, the atherogenic protein comprises OxLDL. In some embodiments, the atherogenic protein comprises OxLDL containing at least 60 substituted lysine residues per apo B-100 moiety. In some embodiments, the acute phase reactant is C-reactive protein and/or fibrinogen. In some embodiments, the anti-atherogenic protein comprises HDL. In some embodiments, an immunological assay is used to obtain the level of atherogenic protein. In some embodiments, the immunological assay uses one or more monoclonal antibodies each having an affinity for the atherogenic protein of at least about 5×10⁸ M⁻¹. In some embodiments, the immunological assay uses at least one of the following monoclonal antibodies to obtain the level of atherogenic protein: mAb-4E6 produced by hybridoma Hyb4E6 deposited with the BCCM (Belgian Coordinated Collections of Microorganisms) under deposit accession number LMBP 1660 CB, mAb-1H11 produced by hybridoma Hyb1H11 deposited with the BCCM under deposit accession number LMBP 1659 CB, and mAb-8A2 produced by hybridoma Hyb8A2 deposited with the BCCM under deposit accession number LMBP 1661 CB (the cell lines were deposited on Apr. 24, 1997). In some embodiments, the atherogenic protein comprises an atherogenic low density lipoprotein, step (a) is conducted using an immunological assay, and the immunological assay can detect the presence of the atherogenic low density lipoprotein in undiluted human plasma in a concentration of 0.02 milligrams/deciliter.

The present invention allows significantly better discrimination between those who have coronary artery disease and those who do not but all of whom are asymptomatic. This can be better understood by considering the following.

If a group of people (i.e., a population) who are asymptomatic for coronary artery disease are tested (for example, by assaying one or more samples from each person to measure one or more biochemical markers and then comparing the assay results to one or more respective predetermined cut-points for the one or more markers), with a typical test, some of those who have coronary artery disease will be deemed to have the disease (“true positives”) and some who have the disease will be deemed not to have the disease (“false negatives”). Similarly, some of those who do not have the disease will be deemed to have the disease (“false positives”) and some who do not have the disease will be deemed not to have the disease (“true negatives”). The “sensitivity” of the test (i.e., the true positive rate) is the percentage of those who do have the disease who (based on the test) are deemed to have the disease (i.e., the number of true positives divided by the total of the number of true positives plus the number of false negatives). The “specificity” of the test (i.e., the true negative rate) is the percentage of those who do not have the disease who (based on the test) are deemed not to have the disease (i.e., the number of true negatives divided by the total of the number of true negatives plus the number of false positives). See Shultz E K, “Clinical Interpretation Of Laboratory Procedures,” Chapter 14 in Teitz, Fundamentals of Clinical Chemistry, Burtis C A, Ashwood E R (eds.), 4th edition 1996, W.B.Saunders Company, pages 192-199.

A perfect test would have perfect accuracy. Thus, for individuals who have the disease, the test would indicate only positive test results and would not report any of those individuals as being negative (there would be no false negatives). In other words, the sensitivity of the test would be 100%. On the other hand, for individuals who did not have the disease, the test would indicate only negative test results and would not report any of those individuals as being positive (there would be no false positives). In other words, the specificity would be 100%. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491.

Changing the cut-point (or threshold value) of a test or assay usually changes the sensitivity and specificity but in a qualitatively inverse relationship. For example, if the cut-point is lowered, more individuals in the population tested will typically have test results over the cut-point or threshold value. If individuals who have test results above the cut-point are reported as having the disease for which the test is being run, lowering the cut-point will cause more individuals to be reported as having positive results (i.e., that they have the disease). Thus, a higher proportion of those who have the disease will be indicated by the test to have it. Accordingly, the sensitivity (true positive rate) of the test will be increased. However, at the same time, there will be more false positives because more people who do not have the disease (i.e., people who are truly “negative”) will be indicated by the test to have analyte values above the cut-point and therefore will be reported as positive (i.e., to have the disease) rather than being correctly deemed based on the test to be negative. Accordingly, the specificity (true negative rate) of the test will be decreased. Similarly, raising the cut-point will tend to decrease the sensitivity and increase the specificity. Therefore, in assessing the accuracy and usefulness of a proposed medical test, assay, or method for assessing a patient's condition, one should always take both sensitivity and specificity into account and be mindful of what the cut-point is at which the sensitivity and specificity are being reported because sensitivity and specificity may vary significantly over the range of cut-points.

There is, however, an indicator that allows representation of the sensitivity and specificity of a test, assay, or method over the entire range of cut-points with just a single value. That indicator is derived from a Receiver Operating Characteristics (“ROC”) curve for the test, assay, or method in question. See, e.g., Shultz, “Clinical Interpretation Of Laboratory Procedures,” chapter 14 in Teitz, Fundamentals of Clinical Chemistry, Burtis and Ashwood (eds.), 4th edition 1996, W.B.Saunders Company, pages 192-199; and Zweig et al., “ROC Curve Analysis: An Example Showing The Relationships Among Serum Lipid And Apolipoprotein Concentrations In Identifying Patients With Coronory Artery Disease,” Clin. Chem., 1992, 38(8): 1425-1428.

An ROC curve is an x-y plot of sensitivity on the y-axis, on a scale of zero to one (i.e., 100%), against a value equal to one minus specificity on the x-axis, on a scale of zero to one (i.e., 100%). In other words, it is a plot of the true positive rate against the false positive rate for that test, assay, or method. To construct the ROC curve for the test, assay, or method in question, patients are assessed using a perfectly accurate or “gold standard” method that is independent of the test, assay, or method in question to determine whether the patients are truly positive or negative for the disease, condition, or syndrome (for example, coronary angiography is a gold standard test for the presence of coronary atherosclerosis). The patients are also tested using the test, assay, or method in question, and for varying cut-points, the patients are reported as being positive or negative according to the test, assay, or method. The sensitivity (true positive rate) and the value equal to one minus the specificity (which value equals the false positive rate) are determined for each cut-point, and each pair of x-y values is plotted as a single point on the x-y diagram. The “curve” connecting those points is the ROC curve.

The area under the curve (“AUC”) is the indicator that allows representation of the sensitivity and specificity of a test, assay, or method over the entire range of cut-points with just a single value. The maximum AUC is one (a perfect test) and the minimum area is one half. The closer the AUC is to one, the better is the accuracy of the test. Thus, one way to consider the superiority of the present method (using, e.g., an atherogenic protein and an acute phase reactant) as compared to, for example, using a non-atherogenic protein and an acute phase reactant, is the expected increase in AUC obtainable with the present invention. Thus, it is believed that the present invention would show an increase in AUC of at least 5%, desirably of at least 10%, and preferably of at least 15% if a properly constructed comparative test were performed.

There is another way to understand the advantages of the present method. Consider a situation where there is a gold standard test to determine whether individuals in the population have or do not have the disease (e.g., angiography for coronary artery disease) and where the method in question (e.g., a new method whose utility is being evaluated) uses only a single marker (e.g., total cholesterol) to determine whether an individual has the disease of interest (e.g., coronary artery disease). An x-y (two-dimensional) graph is employed, and the value of the marker is plotted on the x-axis (e.g., total cholesterol values, in milligrams per deciliter, which values increase moving along the x-axis away from the origin). For each marker value along the x-axis (e.g., total cholesterol level), the number of people in the population who have the disease (as determined by the gold standard method) and have that marker value is plotted on the y-axis. These x-y points are then connected, thereby forming a frequency polygon (or curve) of people who have the disease (this curve represents only the true positives in the population). (A frequency polygon is analogous to a histogram, which is a graph that uses vertical bars of different heights to represent frequencies for the different bands or classes of the x variable.) In the same way, for each marker value, the number of people in the population who do not have the disease (as determined by a gold standard method) and have that marker value is plotted on the y-axis. These x-y points are then connected, thereby forming a frequency polygon (or curve) of people who do not have the disease (this curve represents only true negatives in the population).

If the method in question is able to perfectly discriminate between those individuals who do not have the disease (the left or true negatives curve) and those who do (the right or true positives curve), the two curves will not overlap (intersect). On the other hand, if the test or method cannot perfectly discriminate between the two sub-populations, there will be overlap (i.e., an intersection): the right end of the left curve will overlap the left end of the right curve. In other words, there will be a range of x-axis values of the marker (e.g., cholesterol level) for which an individual being tested using the method in question cannot be deemed to be either positive (i.e., to have the disease) or negative (i.e., to not have the disease). If an individual having the disease is deemed by the method in question not to have the disease, that is a false negative; if an individual not having the disease is deemed by the test in question to have the disease, that is a false positive. The farther the means for the two curves are and/or the smaller the standard deviations for each curve, the smaller the overlap is likely to be and the less likely there will be false positives or false negatives.

The analysis is the same regardless of how many markers are used by the method in question. For example, if a method employs two markers and the readings for each are combined to produce a single value (e.g., by normalizing each of the two values and multiplying the two normalized values to produce a single value), that single value may be graphed on the x-axis and the frequency graphed on the y-axis (just as before). If the method employs the two markers separately, a three-dimensional (i.e., x-y-z) graph may be used, with one marker being graphed on the x-axis, the other marker being graphed on the y-axis, and the frequency being graphed on the z-axis. In that case, each of the negative and positive polygons (curves) would be three-dimensional rather than two-dimensional. If the method in question perfectly discriminated between those having the disease and those not having the disease, the two three-dimensional curves would not overlap. On the other hand, the two three-dimensional curves would overlap if the method were less than perfect, and the method would not be able to deem (classify) an individual as having or as not having the disease if his or her two marker values placed him or her in the region (volume) of overlap.

One skilled in the art will understand that regardless of the number of markers used, the smaller the overlap (between the true negatives curve and the true positives curve), the better the method. For people in the general population who are asymptomatic for coronary artery disease, with a method using C-reactive protein and total cholesterol (e.g., U.S. Pat. No. 6,040,147), the area of overlap is expected to be approximately 25-40%. In other words, the area of overlap represents approximately 25-40% of the total area of both curves (the total area of both curves represents the population of all asymptomatic people). With the method of the present invention using C-reactive protein and an atherogenic protein such as OxLDL, the area of overlap is expected to be reduced to approximately 20-30%. With a method using C-reactive protein and the total cholesterol:HDL ratio (e.g., U.S. Pat. No. 6,040,147), the area of overlap is expected to be approximately 20-30%. With the method of the present invention using C-reactive protein, an atherogenic protein such as OxLDL, and HDL, the area of overlap is expected to be reduced to approximately 15-25%. These seemingly small percentage reductions in overlap when using the method of this invention are highly significant, both for the individuals involved (who would otherwise not be detected as having coronary artery disease until it was perhaps to late) as well as for society in general.

It is estimated that about 25-30% of the general population has asymptomatic accelerated atherosclerosis. Conservatively assuming that 20% of the general population has asymptomatic coronary artery disease, with a total 2004 United States population of about 290 million people, approximately 58 million people in the United States have asymptomatic coronary artery disease. Even a modest reduction of just a few percent in overlap would result in detecting at least an additional 1 to 2 million people as having coronary artery disease, and those individuals who learned they had the disease could try to modify their lifestyles (e.g., diet, exercise) and/or be treated with drugs (e.g., statins) to try to arrest or reverse the disease. If such early intervention prevented just a small fraction of them from having an acute myocardial infarction or from having to undergo various operations (e.g., coronary artery bypass graft surgery), the savings in lives and money would be substantial.

According to the American Heart Association, the total direct and indirect cost of coronary heart disease in the United States in 2004 is estimated to be about $133 billion (for comparison, the cost in the United States for 2004 is estimated to be about $368 billion for all cardiovascular disease), of which about $66 billion is direct cost (i.e., the cost of hospitals, nursing homes, medical professionals, drugs and medical durables, and home health care) and of which about $67 billion is indirect cost (i.e., the cost of lost productivity from morbidity and mortality). The number of people in the United States in 2001 diagnosed as having coronary heart disease was estimated to be about 13.2 million. In the United States, more than 400,000 individuals each year have coronary artery bypass graft surgery, and it is the most frequently performed major operation in the United States. The annual cost for these operations is at least $10 billion and the increase in life span averages about 5 years. The numbers are similar for angioplasty.

It should be apparent that it would be far more advantageous for society and for the individuals involved if coronary artery disease could be detected in just a small fraction of the tens of millions of asymptomatic individuals so that at least some of them could take appropriate action and thereby slow or reverse progression of the disease. Most advantageously, the present invention facilitates significantly improved detection of coronary artery disease in asymptomatic people, thereby benefiting the individuals involved and society.

### DETAILED DESCRIPTION OF THE INVENTION

The present invention concerns a method of making an assessment of the likelihood that a human patient who is asymptomatic for coronary artery disease does in fact have the disease. “Coronary artery disease” is a stage of arteriosclerosis (pathological hardening or thickening of the arterial wall) involving fatty deposits (atheromas or arterial plaque) inside the coronary arteries (there are two main coronary arteries that supply heart muscle, the left coronary artery and the right coronary artery).

In the present method, the level of an atherogenic protein in a sample from the patient is obtained and the level of an acute phase reactant in a sample from the patient is obtained.

The atherogenic protein may be any atherogenic protein, although as will be understood by one skilled in the art, some may be more desirable to use (e.g., because their values for patients are easier to obtain, because in combination with the particular acute phase reactant(s) employed, they provide better discrimination, i.e., reduced overlap). As used herein, an “atherogenic protein” is a protein that causes or participates in the formation of plaque in the lining of the arteries or, in other words, participates in the atherosclerotic disease process. The atherogenic proteins used will desirably be those atherogenic proteins first appearing in the body earlier in the coronary artery disease process (e.g., oxidized low density lipoprotein or OxLDL) rather than those first appearing later in the disease process (MDA-modified LDL may be such a substance). Thus, MDA-modified LDL (e.g., MDA-modified LDL containing at least 60 substituted lysine residues per apo B-100 (apolipoprotein B-100) moiety) may be useful in some cases. Preferred atherogenic proteins comprise OxLDL and particularly OxLDL containing at least 60 substituted lysine residues per apo B-100 (apolipoprotein B-100) moiety. The atherogenic protein may be a mixture of two or more atherogenic proteins.

The acute phase reactant may be any acute phase reactant, although as will be understood by one skilled in the art, some may be more desirable to use (e.g., because their values for patients are easier to obtain, because in combination with the particular atherogenic protein(s) employed, they provide better discrimination, i.e., reduced overlap). An “acute phase reactant” is a protein whose level in the body increases (i.e., a positive acute phase reactant) or decreases (i.e., a negative acute phase reactant) as a result of an inflammatory stimulus. Acute phase reactants are also referred to as “acute phase proteins.” Although most acute phase reactants are synthesized by hepatocytes, some are produced by other cells, such as monocytes, endothelial cells, fibroblasts, and adipocytes. Some positive acute phase reactants increase by about half, up to several-fold, their normal levels as part of the inflammatory (acute phase) response, while others can increase a thousand-fold over their normal levels. It is believed that the production of negative acute phase reactants decreases during the inflammatory response to allow the body to direct more of its synthesis capacity to making the positive acute phase reactants. The acute phase reactant may be a mixture of two or more acute phase reactants, although such a mixture preferably would contain only positive acute phase reactants or only negative acute phase reactants.

In humans, the positive acute phase reactants include the major acute phase reactants, namely, serum amyloid A (SAA) and C-reactive protein (CPR), as well as a number of minor acute phase reactants: (i) complement proteins: C2, C3, C4, C5, C9, C1 inhibitor, and C4 binding protein; (ii) coagulation proteins: fibrinogen and von Willebrand factor; (iii) proteinase inhibitors: alpha1-antitrypsin, alpha1-antichymotrypsin, alpha2-antiplasmin, heparin cofactor II, plasminogen activator inhibitor I; metal binding proteins haptoglobin, hemopexin, ceruloplasmin, manganese superoxide dismutase; and (iv) other proteins: alpha1-acid glycoprotein, heme oxygenase, ferritin, mannose-binding protein, leukocyte protein I, lipoprotein (a), lipopolysaccharide-binding protein. In humans, the negative acute phase reactants include albumin, pre-albumin, transferin, apo A-I (apolipoprotein A-I), apo A-II (apolipoprotein A-II), HDL (high density lipoprotein), HS glycoprotein, inter-alpha-trypsin inhibitor, and histidine-rich glycoprotein.

The preferred positive acute phase reactants used herein are C-reactive protein, serum amyloid A, von Willebrand factor, ferritin, and fibrinogen, and the preferred negative acute phase reactant used herein are albumin, apo A-I, apo A-II, and HDL. The most preferred acute phase reactants are C-reactive protein and fibrinogen.

Broadly speaking, acute phase reactants are markers of systemic inflammation and are not markers of inflammation of or injury to any specific organ, nor are they specific markers of any particular disease. Elevated levels of acute phase reactants are associated with a wide variety of diseases and conditions (e.g., infections with gram-positive and gram-negative organisms, rheumatoid arthritis, abdominal abscesses, multiple sclerosis, tuberculosis, burns, and patients with surgical trauma).

The anti-atherogenic protein, which is optionally used, may be any anti-atherogenic protein, although as will be understood by one skilled in the art, some may be more desirable to use (e.g., because their values for patients are easier to obtain, because in combination with the particular atherogenic protein(s) and acute phase reactant(s) employed, they provide better discrimination, i.e., reduced overlap). As used herein, an “anti-atherogenic protein” is a protein that directly or indirectly hinders the formation of plaque in the lining of the arteries or, in other words, directly or indirectly hinders the atherosclerotic disease process. A preferred anti-atherogenic protein is HDL. The anti-atherogenic protein may be a mixture of two or more anti-atherogenic proteins.

A few substances may be in more than one category. For example, certain proteins may be negative acute phase reactants as well as being anti-atherogenic proteins (e.g., HDL and apo A-I). In such cases, the substance will be considered to be in just one category, since its being in more than one category would not add new information for making the determination of whether the asymptomatic individual had or did not have coronary artery disease.

There are seven possible combinations of atherogenic protein, acute phase reactant, and optional anti-atherogenic protein that may be used (e.g., arithmetically, graphically): (i) atherogenic protein and acute phase reactant used individually, without any anti-atherogenic protein being used; (ii) atherogenic protein and acute phase reactant used together, without any anti-atherogenic protein being used; (iii) atherogenic protein and acute phase reactant used together, with anti-atherogenic protein being used individually; (iv) atherogenic protein being used individually, with acute phase reactant and anti-atherogenic protein being used together; (v) atherogenic protein and anti-atherogenic protein used together, with acute phase reactant being used alone; (vi) all three of atherogenic protein, acute phase reactant, and anti-atherogenic protein used together; and (vii) each of atherogenic protein, acute phase reactant, and anti-atherogenic protein used individually.

The seven combinations of atherogenic protein and acute phase reactant and optional anti-atherogenic protein are summarized in Table I, below. Within each combination, the substances marked “used together” are used with each other and the substance(s) marked “used alone” are used individually. Also shown for each of the seven combinations are the total number of values and the total number of cut-points needed for making the comparison (for each combination, the number of values will be the same as the number of cut-points) and the ordinal number(s) (from first to thirteenth) of the values and cut-point(s) used. For example, in making the assessment, the comparisons that can be made are (i) of the first value (which is related to the level of the atherogenic protein) to the first cut-point (which is related to the atherogenic protein used) and of the second value (which is related to the level of the acute phase reactant) to the second cut-point (which is related to the acute phase reactant used), and/or (ii) of the third value (which is related to both the level of the atherogenic protein and the level of the acute phase reactant) to the third cut-point (which is related to both the atherogenic protein and the acute phase reactant), and/or (iii) of the fourth value (which is related to both the level of the atherogenic protein and the level of the acute phase reactant) to the fourth cut-point (which is related to both the atherogenic protein and the acute phase reactant) and of the of the fifth value (which is related to the level of the anti-atherogenic protein) to the fifth cut-point (which is related to the anti-atherogenic protein), etc.

TABLE I
THE SEVEN COMBINATIONS OF ATHEROGENIC PROTEIN,
ACUTE PHASE REACTANT, AND OPTIONAL
ANTI-ATHEROGENIC PROTEIN

| | (i) | (ii) | (iii) | (iv) | (v) | (vi) | (vii) |
|---|---|---|---|---|---|---|---|
| Atherogenic | used | used | used | used | used | used | used |
| protein | alone | together | together | alone | together | together | alone |
| Acute phase | used | used | used | used | used | used | used |
| reactant | alone | together | together | together | alone | together | alone |
| Anti-atherogenic | — [not | — [not | used | used | used | used | used |
| protein | used] | used] | alone | together | together | together | alone |
| Total values and | 2 | 1 | 2 | 2 | 2 | 1 | 3 |
| total cut-points | | | | | | | |
| each needed | | | | | | | |
| Ordinal numbers | 1^{st} and | 3^{rd} | 4^{th} and | 6^{th} and | 8^{th} and | 10^{th} | 11^{th}, 12^{th}, |
| of the values and | 2^{nd} | | 5^{th} | 7^{th} | 9^{th} | | and 13^{th} |
| the cut-points | | | | | | | |

“Obtain,” “obtaining,” “obtained,” and the like (e.g., as in “obtaining the level of an atherogenic protein in a sample from the patient,” “obtaining the level of an acute phase reactant in a sample from the patient,” “optionally obtaining the level of an anti-atherogenic protein in a sample from the patient,” and “obtaining a cut-point”) should be broadly understood to mean all methods of an entity's directly or indirectly coming into possession of the thing in question or of the entity's having it conveyed on the entity's behalf by one or more third parties to a fourth party without the entity ever being in possession of it (e.g., samples and information of all types, including data and assessments). Thus, an entity's obtaining a sample includes each of the following: (i) the entity's taking (or having one or more third parties take) a sample from the patient and (ii) the entity's inspecting (or having one or more third parties inspect) a library of samples to extract a previously catalogued samples. Similarly, an entity's obtaining the level of a substance in a sample from the patient includes each of the following: (i) the entity's analyzing (or having one or more third parties analyze) a sample (regardless of who takes the sample from the patient or how or when it is or was taken) to determine the level of the substance and (ii) the entity's consulting (or having one or more third parties consult) a database or library of substances to extract previously obtained level of substance information. An entity's obtaining a cut-point includes each of the following: (i) the entity's analyzing (or having one or more third parties analyze) information and/or samples (regardless of who obtains the information and/or samples or how or when obtained) to determine the cut-point and (ii) the entity's consulting (or having one or more third parties consult) a database or library to extract a previously obtained cut-point (regardless of who extracts the cut-point or how or when previously obtained). (As used herein, the words “includes,” “including,” and the like should be broadly understood as, for example, introducing a non-limiting and non-exhaustive list of exemplary items.)

Thus, for example, a clinical chemistry laboratory may obtain the level of a substance when it performs an assay on a sample and determines the level (concentration) of the substance in the sample. A clinical chemistry laboratory may obtain a cut-point when it analyzes data for a population to determine the value at which a risk increases significantly or it may obtain the cut-point when it consults the medical literature or its own database and extracts the cut-point from the literature or the database. A medical doctor may directly obtain the level(s) of one or more substances in a sample when the laboratory reports the level(s) to him or her, or he or she may indirectly obtain the level(s) of one or more substances in a sample when the laboratory reports to him or her that individuals having such or similar levels of substance(s) are (or are not) at increased (or decreased) risk for coronary artery disease (even if one or more of the level(s) are not explicitly reported) or reports to him or her that the individual in question is at an increased risk for coronary artery disease because of the level(s) of substance(s) for that individual (even if one or more of the level(s) are not explicitly reported). A medical doctor may directly obtain a cut-point when he or she analyzes data for a population to determine the value at which a risk increases significantly or when the laboratory reports the cut-point to him or her. A doctor may indirectly obtain a cut-point when the laboratory reports to him or her that individuals having a level of substance found in the patient's sample are (or are not) at increased (or decreased) risk for coronary artery disease (even if the patient's level(s) and/or the cut-point(s) are not explicitly reported).

A sample may be any sample that allows the benefits of this invention to be achieved. The sample from which the level of atherogenic protein is obtained, the sample from which the level of acute phase reactant is obtained, and the sample from which the level of anti-atherogenic protein is optionally obtained may be the same sample from the patient or different samples from the patient. The one or more samples may comprise solid, liquid, and/or gas. For example, samples may comprise tissue or fractions or derivatives thereof (e.g., tissue extract) or whole blood or other body fluids or fractions or derivatives thereof (e.g., plasma, serum). Preferably, a single blood sample from the patient is taken and processed by automated laboratory equipment to perform the two or more analyses required to obtain the levels of atherogenic protein, acute phase reactant, etc.

Typically, the “sample from the patient” will be a fluid sample, typically whole blood or a fluid derived from whole blood (such as plasma or serum). Fluid samples (particularly whole blood, plasma, or serum), as opposed to tissue samples, have the advantage of being easily and quickly obtained and tested, which is particularly important in a clinical setting where time may be of the essence. Also, clinicians are accustomed to withdrawing fluid samples (particularly blood) from patients, and some of the markers may not be present or may not be present in sufficient quantities in tissue samples.

Whole blood may contain substances, e.g., cells, that interfere with the tests used in the method of the invention and, therefore, whole blood is a less preferred sample. The preferred sample is plasma, which is whole blood from which the cells (red blood cells, white blood cells, and platelets) have been removed, e.g., by centrifugation. Serum is plasma from which the fibrinogen has been removed (e.g., by causing clotting and then removing the clotted material) and is less preferred than plasma for use as a sample.

In addition to obtaining the levels of the substances of interest, cut-points related to those substances are obtained. A cut-point (or threshold value) is a value for a given parameter (e.g., analyte) that divides a positive indication from a negative indication. For example, an HDL value below 40 milligrams per deciliter (mg/dL) in adults is often considered to put them at risk for coronary artery disease. In that case, 40 mg/dL is the cut-point between being at risk (if below the cut-point) or not being at risk (if at or above the cut-point), at least with respect to that one risk factor. Some workers in the diabetes field believe that the disease is indicated by a blood glucose value of 126 milligrams per deciliter or higher after an overnight fast or by a random blood glucose (not fasting) value exceeding 200 milligrams per deciliter. For those workers, a fasting blood glucose value of 126 mg/dL is the cut-point between an indication of diabetes (if at or above the cut-point) and an indication of no diabetes (if below the cut-point).

Because there are seven possible combinations of atherogenic protein, acute phase reactant, and optional anti-atherogenic protein that may be used (e.g., arithmetically, graphically), any of seven sets of cut-points may be used: (i) a first cut-point related to the atherogenic protein and a second cut-point related to the acute phase reactant, (ii) a third cut-point related to the atherogenic protein and the acute phase reactant, (iii) a fourth cut-point related to the atherogenic protein and the acute phase reactant and a fifth cut-point related to the anti-atherogenic protein, (iv) a sixth cut-point related to the atherogenic protein and a seventh cut-point related to the acute phase reactant and the anti-atherogenic protein, (v) an eighth cut-point related to the atherogenic protein and the anti-atherogenic protein and a ninth cut-point related to the acute phase reactant, (vi) a tenth cut-point related to the atherogenic protein, the acute phase reactant, and the anti-atherogenic protein, and/or (vii) an eleventh cut-point related to the atherogenic protein, a twelfth cut-point related to the acute phase reactant, and a thirteenth cut-point related to the anti-atherogenic protein. As will be clear to one skilled in the art, “related to” should be broadly understood to include any type of relationship, whether quantitative or qualitative.

The cut-points used will depend on which atherogenic protein, acute phase reactant, and optional anti-atherogenic protein are employed (as well as on whether any further substance(s) not in any of those categories is or are employed), what assays are employed to determine their levels, and how the levels are used (e.g., atherogenic protein and acute phase reactant used together, with anti-atherogenic protein being used individually). For example, if atherogenic protein is being used by itself and the particular atherogenic protein employed is one that in individuals with coronary artery disease is typically present at levels of at least 2 mg/dL of plasma and is not detected in individuals without coronary artery disease (except for background or noise levels of 0.6 mg/dL or less, depending on the particular assay employed), the cut-point, which is “related to” the atherogenic protein, may be established as 1.9 mg/dL.

It will be apparent that a cut-point related to just one of the three substances (e.g., a cut-point related to the atherogenic protein, or a cut-point related to the acute phase reactant, or a cut-point related to the anti-atherogenic protein) does not need to be obtained if that one substance is being used in combination with one of the other substances. For example, neither the first cut-point related to atherogenic protein nor the second cut-point related to acute phase reactant needs to be obtained (or used) if the levels of those two substances are being used to obtain a single value based on those two levels; however, in that case, the third cut-point related to the atherogenic protein and acute phase reactant would be needed.

After the patient's levels of atherogenic protein and acute phase reactant (and optionally of anti-atherogenic protein) and required one or more cut-points are obtained, at least one of the following comparisons is made: (i) a comparison to the first cut-point of a first value related to the level of the atherogenic protein and a comparison to the second cut-point of a second value related to the level of the acute phase reactant, (ii) a comparison to the third cut-point of a third value related to the levels of the atherogenic protein and acute phase reactant, (iii) a comparison to the fourth cut-point of a fourth value related to the levels of the atherogenic protein and acute phase reactant and a comparison to the fifth cut-point of a fifth value related to the level of the anti-atherogenic protein, (iv) a comparison to the sixth cut-point of a sixth value related to the level of the atherogenic protein and a comparison to the seventh cut-point of a seventh value related to the levels of the acute phase reactant and anti-atherogenic protein, (v) a comparison to the eighth cut-point of an eighth value related to the levels of the atherogenic protein and anti-atherogenic protein and a comparison to the ninth cut-point of a ninth value related to the level of the acute phase reactant, (vi) a comparison to the tenth cut-point of a tenth value related to the levels of the atherogenic protein, acute phase reactant, and anti-atherogenic protein, and (vii) a comparison to the eleventh cut-point of an eleventh value related to the level of the atherogenic protein, a comparison to the twelfth cut-point of a twelfth value related to the level of the acute phase reactant, and a comparison to the thirteenth cut-point of a thirteenth value related to the level of the anti-atherogenic protein. The term “comparison” should be understood broadly to include any type of comparison, whether preliminary or final, whether mathematical or otherwise, and whether qualitative or quantitative.

The first, second, third, fourth, fifth, sixth, seventh, eighth, ninth, tenth, eleventh, twelfth, and thirteenth values used in making the comparisons are related to the levels of the various substances (i.e., the atherogenic protein, acute phase reactant, and optional anti-atherogenic protein) obtained for the patient, alone or in combination. Even though the first value, sixth value, and eleventh value are each related to the level of atherogenic protein, they may be the same or different (because, e.g., the methodology used to determine each of the values may differ, depending on what other values are being used to make the assessment). Similarly, even though the third value and fourth value are each related to the levels of both atherogenic protein and acute phase reactant, they may differ. Similarly, even though the second value, ninth value, and twelfth value are each related to the level of acute phase reactant, they may differ. Similarly, even though the fifth value and thirteenth value are each related to the level of anti-atherogenic protein, they may differ.

Just as the values that are related to the level of the same substance may differ, the cut-points that are related to the substance itself may differ. For example, even though the first cut-point, sixth cut-point, and eleventh cut-point are each related to the atherogenic protein, they may be the same or different, depending, for example, on what other substances are being used to make the assessment.

As will be understood by one skilled in the art, “related to” should be broadly understood to refer to any type of relationships of the values to the levels that allow the advantages of this invention to be realized, including quantitative and qualitative relationships (e.g., arithmetic, graphical). For example, the levels obtained for a patient may be mathematically manipulated alone or in groups of two (e.g., the atherogenic protein level for a patient multiplied by the acute phase reactant level for that patient, thereby to obtain a single value that is related to the levels of atherogenic protein and acute phase reactant) or in groups of three (e.g., the normalized atherogenic protein level for a patient multiplied by the normalized acute phase reactant level for that patient divided by the normalized anti-atherogenic protein level for the patient, thereby to obtain a single value that is related to the levels of atherogenic protein, acute phase reactant, and anti-acute phase reactant). Thus, each level may be used as is (e.g., after being obtained by the respective assay) to obtain the value related to the level or a derivative of the level may be used to obtain the value related to the level. For example, some or all of the levels may be normalized (e.g., divided by the mean or median for the entire population or sub-population), or a power of the level (e.g., the square, the cube root), whether or not normalized, may be used, or the reciprocal of any of the them may be used. One or more of the levels (or one or more derivatives of them) may be ranked (e.g., in quartiles) and the rank(s) (or one or more derivatives of them) used.

One or more other substances (i.e., substances that are not within any of the three categories of atherogenic protein, acute phase reactant, and optional anti-atherogenic protein) may also be used in addition to the two necessary (atherogenic protein and acute phase reactant) and optional (anti-atherogenic protein) categories of substances, either in combination (e.g., the level(s) of the other substances are mathematically combined with the levels of one or more substances within the three categories) or alone (e.g., the level(s) of the other substances are not mathematically combined with the levels of one or more substances within the three categories). Preferably, however, only substances within the categories of atherogenic protein, acute phase reactant, and optional anti-atherogenic protein will be used.

A value based on the level of one or more of those three substances is still “related to” the one or more of those three substances even if the value is also based on one or more other substances. For example, a value based on the level of one or more such other substances as well as on the level of the acute phase reactant is still related to the level of the acute phase reactant. As another example, a value based on the level of one or more other substances as well as on the levels of the atherogenic protein, acute phase reactant, and anti-atherogenic protein is still related to the levels of the atherogenic protein, acute phase reactant, and anti-atherogenic protein. If the level of an other substance is used to calculate a value used in the comparison (i.e., the comparison of a value to its respective cut-point), obtaining the respective cut-point will take that other substance into account.

Based on the one or more comparisons (e.g., a comparison to the eleventh cut-point of an eleventh value related to the level of the atherogenic protein obtained for the individual, a comparison to the twelfth cut-point of a twelfth value related to the level of the acute phase reactant obtained for the individual, and a comparison to the thirteenth cut-point of a thirteenth value related to the level of the anti-atherogenic protein obtained for the individual), an assessment is made as to whether the individual is likely to have asymptomatic coronary artery disease.

The assessment need not be made by a medical professional. The term “medical professional” includes medical doctors, doctors of osteopathy, nurse practitioners, and anyone else permitted under applicable law to make diagnoses and/or prescribe and/or administer courses of medical treatment (e.g., involving surgery and/or medicine). The terms “assess,” “assessing,” “assessment,” and the like should be understood broadly to include any type of preliminary or final determination, appraisal, consideration, evaluation, or the like, whether or not rising to the level of a medical diagnosis. A clinical chemistry laboratory providing (e.g., to a doctor) the results of laboratory determinations concerning the substances of principal interest (i.e., atherogenic protein, acute phase reactant, and optional anti-atherogenic protein) would be considered to have made an assessment merely by including in what is provided (e.g., in the notification of the results or other report) an indication that individuals having certain levels have (or may have, or do not have, etc.) an increased risk of coronary artery disease. The laboratory would not be including such a message in the notification unless it had made some type of comparison.

A clinical laboratory or other person or entity may also facilitate the assessment by a medical professional (i.e., the assessment of the likelihood that a patient who is asymptomatic for coronary artery disease in fact has the disease). In that case, the clinical chemistry laboratory or other person or entity would directly or indirectly obtain the appropriate levels in one or more samples from a patient, directly or indirectly obtain the one or more required cut-points, directly or indirectly provide to the medical professional one or more values related to the substance levels for that patient, and directly or indirectly provide to the medical professional the appropriate cut-points so that the medical professional could make the assessment.

“Provide,” “providing,” “provided,” and the like (e.g., as in “providing to the medical professional at least one of . . . a first value related to the level of the atherogenic protein and a second value related to the level of the acute phase reactant . . . ,” “providing to the medical professional the appropriate one or more of the cut-points to permit the medical professional to assess whether the patient is likely to have asymptomatic coronary artery disease based on at least one of the following . . . ”) should be broadly understood to mean all methods of an entity's directly or indirectly providing the thing in question (including the entity's having it conveyed on the entity's behalf by one or more third parties to the recipient (e.g., the medical professional) without the entity ever being in possession of it (e.g., samples and information of all types, including data and assessments). For example, an entity's “providing to the medical professional the appropriate one or more of the cut-points” includes the entity's giving, making available, imparting, or the like (or having one or more third parties give, make available, impart, or the like) the appropriate one or more of the cut-points to the medical professional, regardless of the source of the one or more cut-points and regardless of when the cut-points were obtained.

Each of the atherogenic protein and anti-atherogenic protein will often comprise lipoproteins. Lipoproteins are multicomponent complexes of protein and lipids. Each type of lipoprotein has a characteristic molecular weight, size, chemical composition, density, and physical role. The protein and lipid are held together by noncovalent forces.

Lipoproteins can be classified on the basis of their density as determined by ultracentrifugation. Thus, four classes of lipoproteins can be distinguished: High Density Lipoproteins (“HDL”), Intermediate Density Lipoproteins (“IDL”), Low Density Lipoproteins (“LDL”), and Very Low Density Lipoproteins (“VLDL”).

The purified protein components of a lipoprotein particle are called apolipoproteins (apo). Each type of lipoprotein has a characteristic apolipoprotein composition. In LDL the prominent apolipoprotein protein is apo B-100, which is one of the longest single chain polypeptides known and consists of 4536 amino acids. Of these amino acids, the lysine residues or moieties (there are 356 such lysine residues or moieties) can be substituted or modified by aldehydes (e.g., malondialdehyde).

Oxidation of the lipids in LDL (whether in vitro, e.g., by copper-induced oxidation, or whether in vivo) results in the generation of reactive aldehydes, which can then interact with the lysine residues or moieties of apo B-100. The outcome of this lysine substitution or modification is that the resulting oxidized low density lipoprotein (“OxLDL”), which is also malondialdehyde-modified low density lipoprotein (“MDA-modified LDL”), is no longer recognized by the LDL receptor at the surface of fibroblasts but by scavenger receptors at the surface of macrophages. Holvoet and others report that at least 60 out of the 356 lysines (or lysine residues or moieties) of apo B-100 have to be substituted in order to be recognized by the scavenger receptors. The uptake of such OxLDL by macrophages results in foam cell generation, which is considered to be an initial step in atherosclerosis.

Endothelial cells under oxidative stress (e.g., in acute myocardial infarction patients) and activated blood platelets also produce aldehydes, which interact with the lysine moieties in apo B-100, resulting in the generation of aldehyde-modified LDL that is also recognized by the scavenger receptors. However, the lipids in this aldehyde-modified LDL are not oxidized. Enzymatic activity in macrophages (e.g., myeloperoxidase) results in the oxidation of both the lipid and the protein moieties of LDL. All these pathways result in aldehyde-type modification of the protein moiety of LDL.

Testing the sample(s) for the atherogenic protein, acute phase reactant, and optional anti-atherogenic protein may employ any assays, methodology, and equipment that allow the benefits of this invention to be achieved, e.g., chemical assays and immunological assays, such as competitive and sandwich assays, may be used for the atherogenic protein, acute phase reactant, and anti-atherogenic protein.

In an immunological assay, any antibodies that have suitably high affinity for the target species may be used, and preferably the antibodies are monoclonal antibodies. “High affinity” means an affinity constant (association constant) of at least about 5×10⁸ M⁻¹ (where “M” indicate molarity or moles per liter, and “M⁻¹” indicates reciprocal molarity or liters per mole), desirably of at least about 1×10⁹ M⁻¹, preferably of at least about 1×10¹⁰ M⁻¹, and most preferably of at least about 1×10¹¹ M⁻¹. As used herein, “low affinity” (in contradistinction to high affinity) means an affinity constant (association constant) of less than about 1×10⁷ M⁻¹, desirably less than about 1×10⁶ M⁻¹, and preferably less than about 1×10⁵ M⁻¹. Affinity constants are determined in accordance with the appropriate method described in Holvoet et al., J. Clin. Invest. 1994, 93: 89-98.

The preferred atherogenic proteins comprise OxLDL whose apo B-100 moieties contain at least 60, desirably up to about 90, more desirably up to about 120, preferably up to about 180, more preferably up to about 210, and most preferably possibly up to about 240 substituted lysine residues per apo B-100 moiety. As noted above, in some cases the atherogenic protein may comprise MDA-modified LDL, in which case the MDA-modified LDL would have the same increasingly preferred levels of substitution. The range of lysine substitution will generally be from 60 up to about 240 substituted lysine moieties per apo B-100 moiety and sometimes from 60 up to about 180 substituted lysine moieties per apo B-100 moiety. Antibodies recognizing epitopes present when less than about 60 lysines per apo B-100 moiety are substituted or modified are less specific but are still useful (e.g., they may be used as the secondary antibody in a sandwich Enzyme-Linked Immunosorbent Assay (“ELISA”)).

The preferred antibodies used to detect and determine the levels of OxLDL and MDA-modified LDL are monoclonal antibodies mAb-4E6, mAb-1H11, and mAb-8A2. Their affinity constants for native LDL, MDA-modified LDL, and OxLDL are shown below in Table II (the units are liters per mole, which is the reciprocal of molarity or M⁻¹):

TABLE II

| | | MDA-modified | |
|---|---|---|---|
| Antibody | Native LDL | LDL | OxLDL |
| mAb-4E6 | less than 1 × 10⁶ | 3 × 10¹⁰ | 2 × 10¹⁰ |
| mAb-1H11 | less than 1 × 10⁶ | 3 × 10¹⁰ | less than 1 × 10⁶ |
| mAb-8A2 | 5 × 10⁹ | 1 × 10¹⁰ | 1 × 10¹⁰ |

Monoclonal antibody mAb-4E6 is produced by hybridoma Hyb4E6 deposited at the BCCM under deposit accession number LMBP 1660 CB on Apr. 24, 1997. Monoclonal antibody mAb-1H11 is produced by hybridoma Hyb1H11 deposited at the BCCM under deposit accession number LMBP 1659 CB on Apr. 24, 1997. Monoclonal antibody mAb-8A2 is produced by hybridoma Hyb8A2 deposited at the BCCM under deposit accession number LMBP 1661 CB on Apr. 24, 1997.

The BCCM is the Belgian Coordinated Collections of Microorganisms authorized by the Budapest Treaty Of 28 Apr. 1977 On The International Recognition Of The Deposit Of Microorganisms For The Purpose Of Patent Procedure (“Budapest Treaty”). Its address is c/o The University of Gent, K. L. Ledeganckstraat 35, B-9000 Gent, Belgium.

The three deposits were made at the BCCM on Apr. 24, 1997 under conditions prescribed by the Budapest Treaty and in accordance with The United States Code Of Federal Regulations (see 37 CFR § 1.808) and The United States Patent And Trademark Office's Manual Of Patent Examination (“MPEP”) (see § 2410.01). Issued U.S. Pat. No. 6,309,888 references the same three deposits.

The three preferred antibodies were made using the procedure set forth in detail in U.S. Pat. No. 6,309,888 and International Patent Publications WO 98/59248 and WO 00/14548 (which, as indicated above, are incorporated herein in their entirety for all purposes). Briefly, Balb/c mice were immunized by intravenous and intraperitoneal injection of either OxLDL or MDA-modified LDL. OxLDL was obtained by in vitro incubation of LDL (final apo B-100 concentration 700 μg/mL (micrograms per milliliter)) with copper chloride (final concentration 640 μM) for 16 hours at 37° C. MDA-modified LDL was prepared by incubation of LDL (final apo B-100 concentration 700 μg/mL) with a 0.25 M (molar) MDA solution for 3 hours at 37° C. The numbers of substituted lysines, measured in the TBARS assay, was typically 210 per apo B-100 molecule for OxLDL and 240 for MDA-modified LDL. Hybridomas were obtained by PEG-induced fusion of spleen lymphocytes derived from immunized mice with P3-X63/Ag-6.5.3 myeloma cells according to standard techniques (Holvoet et al., J. Clin. Invest. 1994; 93: 89-98). The screening for hybridomas producing specific antibodies was performed with ELISA using microtiter plates coated with MDA-modified LDL or copper-oxidized LDL. Three hundred eight hybridomas were obtained after immunization of mice with either OxLDL (211) or MDA-modified LDL (97). Hyb4E6 produced antibodies specific for both MDA-modified and copper-oxidized LDL (mAb-4E6), and Hyb1H11 produced antibodies specific for MDA-modified LDL (mAb-1H11) alone. Mice immunized with LDL in a similar method yielded hybridoma Hyb8A2, which produced antibody mAb-8A2.

In the case of a competitive ELISA for determining the preferred atherogenic proteins, a solid substrate coated with OxLDL or MDA-modified LDL may be contacted for a predetermined period of time with the monoclonal antibody mAb-4E6 and a sample thought or known to contain OxLDL and/or MDA-modified LDL, after which period of time unbound antibody and sample are removed and a binding reaction between antibody and OxLDL and/or MDA-modified LDL bound to the substrate is visualized and/or quantified. Quantification in a competitive ELISA is indirect because the binding between the antibody and the analyte in the sample is not measured but instead the amount of antibody that binds to the known amount of OxLDL or MDA-modified LDL that is coated on (bound to) the substrate is measured. The more antibody bound to the known amount of OxLDL or MDA-modified LDL coated on the substrate, the less analyte there was in the sample.

A typical competitive assay using monoclonal antibody mAb-4E6 is as follows. It is based on the inhibition by copper-oxidized LDL of the binding of mAb-4E6 to the coated wells of microtiter plates. Thus, standard OxLDL (or MDA-modified LDL) and plasma samples are diluted in PBS (phosphate buffered saline) containing 1 mM (millimolar) EDTA, 20 μM (micromolar) Vitamin E, 10 μM butylated hydroxytoluene, 20 μM dipyridamole, and 15 mM theophylline to prevent in vitro LDL oxidation and platelet activation. Equal volumes of diluted purified mAb-4E6 solution (final concentration 7.5 ng/mL (nanograms per milliliter) and of either diluted standard solution or diluted plasma samples (copper-oxidized LDL added as competing ligand at a final concentration ranging from 50 to 500 ng/mL) are mixed and incubated for 30 min at room temperature. Then 200 μL (microliter) aliquots of the mixtures are added to wells coated with MDA-modified LDL or OxLDL. The aliquots are incubated for 2 hours at room temperature. After washing, the wells are incubated for 1 h (hour) with horse-radish peroxidase conjugated rabbit IgG raised against mouse immunoglobulins and washed again. The peroxidase reaction is performed (see Holvoet et al., J. Clin. Invest. 1995, 95: 2611-2619) and the absorbance (A) is read at 492 nm (nanometers). Controls without competing ligand and blanks without antibody may be routinely included. The percent inhibition of binding of mAb-4E6 to the immobilized ligand may be calculated as:

$$\frac{{A}^{492 n m} control - {A}^{492 n m} sample}{{A}^{492 n m} control - {A}^{492 n m} blank}$$

and standard curves may be obtained by plotting the percentage of inhibition against the concentration of competing ligand. The lower limit of detection is 0.020 mg/dL in undiluted human plasma.

In the case of a sandwich ELISA, mAb-4E6 (for MDA-modified LDL and OxLDL) or mAb-1H11 (for MDA-modified LDL) may be bound to a solid substrate and subsequently contacted with a sample to be assayed. After removal of the sample, binding between the specific antibody and OxLDL and/or MDA-modified LDL captured out of the sample can be visualized and/or quantified by detection means. Detection means may be a labeled, less specific secondary antibody that recognizes a different part of the apo B-100 moiety of the captured analyte (e.g., mAb-8A2).

A typical sandwich assay for the preferred atherogenic proteins using monoclonal antibodies mAb-4E6 and mAb-8A2 is as follows. It is based on the binding of immunoreactive material to the wells of microtiter plates coated with the monoclonal antibody mAb-4E6 and the detection of bound immunoreactive material with the use of the monoclonal antibody mAb-8A2 labeled with peroxidase. This version of the ELISA is more suited for use in the clinical laboratory because it overcomes the need to prepare standard solutions of in vitro oxidized and/or aldehyde-modified LDL.

Standard preparations and plasma samples are diluted in PBS containing antioxidants and antiplatelet agents as described above in connection with the competitive ELISA, 180 μL aliquots of 80-fold diluted plasma and of standard solutions containing between 10 and 0.01 nM (nanomolar) of MDA-modified LDL are applied to the wells of microtiter plates coated with mAb-4E6 (200 μL of a 4 μg/mL IgG solution), and incubated for 2 hours at room temperature. After washing, the wells are incubated for 1 hour with horseradish peroxidase conjugated mAb-8A2, IgG (final IgG concentration 65 ng/mL), and washed again. The peroxidase reaction is performed as described above in connection with the competitive ELISA. The absorbance measured at 492 nm will correlate with the log value of the MDA-modified LDL concentration in the range between 1.5 nM and 0.3 nM.

As noted above, any assays, methodology, and equipment may be used provided the benefits of this invention can be achieved. If, for example, an assay employs antibodies, those antibodies may be used in a wide variety of automated immunologic assay systems, which include chemiluminescent immunoassay systems, microparticle enzyme immunoassay systems, fluorescence polarization immunoassay systems, and radioimmunoassay systems.

Applying the method of this invention to the data presented in U.S. Pat. No. 6,309,888 evidences the unexpected advantages of the present invention. As set forth in that patent, a total of 286 individuals associated with the University Hospital Of Leuven either as employees or as individuals who were brought to the emergency department and/or admitted to the Hospital were studied: 105 patients with acute coronary syndromes, 64 patients with stable CAD, and 117 controls.

Individuals were classified as having an acute coronary syndrome (i.e., having an acute stage of coronary artery disease) if they had ischemic chest discomfort with ST-segment elevation or depression of more than 0.5 mm or T wave inversion of more than 1 mm. Of the individuals having an acute stage of coronary artery disease, those whose elevated creatine kinase (CK)-MB levels (and at least 3% of total CK) were present at the time of admission or in samples taken at 6 to 8 hours after admission were classified as having AMI (i.e., acute myocardial infarction). Alternatively, those acute-stage individuals who had no such CK-MB elevations were classified as having unstable angina. Individuals with angiographically documented CAD and no clinical signs of ischemia within the previous month were considered to have stable CAD (i.e., in this case, stable (chronic) angina).

One hundred seventeen individuals (72 males/45 females; mean age=55 years) without a history of atherosclerotic cardiovascular disease were used as controls. They were selected from laboratory and clinical staff of the Hospital and from a population of individuals admitted to the Hospital who did not have a history of atherosclerotic cardiovascular disease.

Venous blood samples were taken in the fasting state in controls and in individuals with stable (chronic) angina. In individuals with acute coronary syndromes, blood samples were taken on admission before the start of treatment. Blood samples were collected on 0.01 M citrate, containing 1 mM EDTA, 20 μM vitamin E, 10 μM butylated hydroxytoluene, 20 μM dipyridamole, and 15 mM theophylline to prevent in vitro LDL oxidation and platelet activation. Blood samples were centrifuged at 3,000 g for 15 minutes at room temperature within 1 hour of collection and the resulting plasma was stored at −20° C. until the assays were performed.

LDL was isolated from pooled plasma of fasting normolipidemic donors by density gradient ultracentrifugation (Havel et al., J. Clin. Invest. 1955, 34: 1345-1353). MDA-modified LDL and copper-oxidized LDL were prepared as described in Haberland et al., Proc. Natl. Acad. Sci USA. 1982, 79: 1712-1716, and Steinbrecher, J. Biol. Chem. 1987, 262(8): 3603-3608, and were used as standards. Characterization of modified LDL involved measurement of thiobarbituric acid reactive substances (“TBARS”), determination of electrophoretic mobility on 1% agarose gels, quantitation of cholesterol and fatty acids by HPLC on a Nova-Pak C-18 reversed-phase column (Waters Associates, Milford, Mass.), quantitation of proteins by Lowry assay, and of phospholipids by enzymatic assay (Biomérieux, Marcy, France). See Holvoet et al., Arterioscler. Thromb. Vasc. Biol. 1998, 18(1): 100-107, and Holvoet et al. J. Clin. Invest. 1995, 95: 2611-2619. Apo B-100 molecules of in vitro MDA-modified LDL and of copper-oxidized LDL contained on average 244 and 210 substituted lysines, respectively. As noted above, although the extent of lysine substitution of in vitro MDA-modified LDL and copper-oxidized LDL is very similar, the lipid moiety in MDA-modified LDL is not oxidized.

A mAb-4E6 based ELISA was used for the quantitation of OxLDL in plasma (see Holvoet et al., Arterioscler. Thromb. Vasc. Biol. 1998, 18(1): 100-107; Holvoet et al., Thromb. Haemost. 1996, 76(5): 663-669; Holvoet et al., Arterioscler. Thromb. Vasc. Biol. 1997, 17(11): 2376-2382; and Holvoet et al., Arterioscler. Thromb. Vasc. Biol. 1998, 18: 415-422). This monoclonal antibody allows the detection of 0.025 mg/dL MDA-modified LDL or copper-oxidized LDL in the presence of 500 mg/dL native LDL. Plasma levels of MDA-modified LDL were measured in a mAb-1H11 based ELISA (see Holvoet et al., J. Clin. Invest. 1995, 95: 2611-2619). This monoclonal antibody allows the detection of 0.025 mg/dL MDA-modified LDL, but not of copper-oxidized LDL, in the presence of 500 mg/dL native LDL. Because the specificities of the two antibodies depend on the extent of protein modification, all lipoprotein concentrations are expressed in terms of protein.

Total cholesterol, HDL cholesterol, and triglycerides were measured by enzymatic methods (Boehringer Mannheim, Meylon, France). LDL cholesterol values were calculated with the Friedewald formula. Troponin I levels were measured on a Beckman ACCESS immunoanalyzer using commercially available monoclonal antibodies (Sanofi, Toulouse, France). C-reactive protein levels were measured in a commercial immunoassay (Boehringer, Brussels, Belgium), and plasma levels of D-dimer were measured in an ELISA as described previously (see Declerck et al., Thromb. Haemost. 1987, 58(4): 1024-1029). As noted above, C-reactive protein is a preferred acute phase reactant used in the present invention. D-dimer is a marker for thrombotic syndromes.

The values obtained are shown in Table III, below (“n” indicates the number of individuals independently known to be in each category).

TABLE III

| | Controls | Stable angina | Unstable angina | AMI |
|---|---|---|---|---|
| | (n = 117) | (n = 64) | (n = 42) | (n = 63) |
| Age | 55 ± 11 | 65 ± 10 | 72 ± 12 | 63 ± 11 |
| Male/female ratio | 72/45 | 53/11 | 28/14 | 42/21 |
| Total cholesterol (mg/dL) | 180 ± 31 | 180 ± 35.3 | 175 ± 36.9 | 175 ± 37.2 |
| LDL cholesterol (mg/dL) | 110 ± 26 | 115 ± 30 | 109 ± 33.4 | 111 ± 32.4 |
| HDL cholesterol (mg/dL) | 49 ± 18 | 37.6 ± 13.2 | 45.2 ± 15.6 | 37.5 ± 9.7 |
| Triglycerides (mg/dL) | 137 ± 66 | 123 ± 46.2 | 103 ± 55.4 | 125 ± 56.7 |
| Oxidized LDL (mg/dL) | 0.85 ± 0.54 | 2.65 ± 0.97 | 3.22 ± 0.85 | 2.97 ± 1.02 |
| MDA-modified LDL (mg/dL) | 0.39 ± 0.15 | 0.46 ± 0.20 | 1.07 ± 0.28 | 1.19 ± 0.43 |
| Troponin I (ng/mL) | 0.0092 ± 0.011 | 0.035 ± 0.12 | 0.37 ± 0.66 | 1.30 ± 1.08 |
| C-reactive protein (mg/dL) | 3.38 ± 1.79 | 6.28 ± 9.0 | 17.4 ± 29.8 | 18.2 ± 35.5 |
| D-dimer (μg/dL) | 166 ± 162 | 299 ± 208 | 367 ± 340 | 602 ± 632 |

Quantitative data represent means ± standard deviations.

Plasma levels of OxLDL were 0.85±0.54 mg/dL (mean±standard deviation) in the 117 controls, and were 3.1-fold higher (p<0.001) in the 64 patients with stable angina pectoris, 3.8-fold higher (p<0.001) in the 42 patients with unstable angina pectoris, and 3.5-fold higher (p<0.001) in the 63 patients with AMI. (For comparison, a group of 79 heart transplant patients without CAD had OxLDL of 1.27±0.061 mg/dL or 1.5-fold higher than the 117 controls and a group of 28 heart transplant patients with stable CAD had OxLDL of 2.49±0.18 mg/dL or 2.9-fold higher than the controls. The reason for the apparent difference between the values for the non-CAD individuals who have had or have not had heart transplants is not known with certainty.)

Plasma levels of MDA-modified LDL were 0.39±0.15 mg/dL in the 117 controls, were only 1.2-fold higher in the 64 patients with stable angina pectoris, but were 2.7-fold higher (p<0.001) in the 42 patients with unstable angina pectoris and 3.1-fold higher (p<0.001) in the 63 AMI patients. (For comparison, a group of 79 heart transplant patients without CAD had MDA-modified LDL of 0.38±0.016 mg/dL or essentially the same as the 117 controls and a group of 28 heart transplant patients with stable CAD had MDA-modified LDL of 0.39±0.038 mg/dL or also essentially the same as the controls.)

Plasma levels of C-reactive protein were 3.38±1.79 mg/dL in the 117 controls, were only 1.9-fold higher in the 64 patients with stable angina, but were 5.1-fold higher (p<0.001) in the 42 patients with unstable angina and 5.4-fold higher in the 63 AMI patients (p<0.001). In agreement with previously published data, C-reactive protein was found to be a marker of acute coronary syndromes (see Muldoon et al., Ryan et al., Oltrona et al., and Liuzzo et al., letters and reply by authors, N. Engl. J. Med. 1995, 332(6): 398-400).

Plasma levels of D-dimer were 166±162 μg/dL in the 117 controls, were only 1.8-fold higher in the 64 patients with stable angina, but were 2.2-fold higher (p<0.001) in the 42 patients with unstable angina and 3.6-fold higher in the 63 AMI patients (p<0.001). In agreement with earlier published data, D-dimer was found to be a marker of acute coronary syndromes (Hoffmeister, Circulation 1995, 91(10): 2520-2527).

For the sub-populations “Control,” “Stable (Chronic) Angina,” and “Acute Coronary Syndromes” (the latter sub-population comprising the 42 individuals with unstable angina and the 63 individuals with acute myocardial infarction), Table 1V, below, shows values calculated in accordance with the present invention, namely, (i) mean OxLDL multiplied by mean C-reactive protein and (ii) mean OxLDL multiplied by mean C-reactive protein divided by mean HDL. For comparison, also shown are the values for the three subpopulations for five comparison cases: (a) mean OxLDL alone, (b) mean CRP alone, (c) mean HDL alone, (d) mean C-reactive protein multiplied by mean total cholesterol (see, e.g., U.S. Pat. No. 6,040,147 and Ridker et al., “C-Reactive Protein Adds To The Predictive Value Of Total And HDL Cholesterol In Determining Risk Of First Myocardial Infarction,” Circulation 1998; 97:2007-2011), and (e) mean C-reactive protein multiplied by mean total cholesterol and divided by mean HDL (id.).

The values and numbers used to calculate them are shown in Table IV without units (the units are irrelevant because all comparisons will be made of values that have the same units. The values for the “Acute Coronary Syndromes” column are the weighed averages (weighted by number of individuals) of the values for unstable angina and acute myocardial infarction. The advantage over the control is calculated for each substance or combination of substances other than HDL as the mean for that substance or combination in the coronary artery disease sub-population (i.e., “Stable Angina” or “Acute Coronary Syndromes”) divided by the respective control mean; however, because the effect of higher HDL is to reduce the risk (HDL is an anti-atherogenic protein), the advantage over the control for HDL is the control mean divided by the mean HDL in the coronary artery disease sub-population.

TABLE IV

| | | | Stable (Chronic) | Acute Coronary |
|---|---|---|---|---|
| | | Control | Angina | Syndromes |
| (i) | OxLDL × CRP | 0.85 × 3.38 = 2.87 | 2.65 × 6.28 = 16.6 | 3.07 × 17.9 = 55.0 |
| | Advantage over control | — | 16.6/2.87 = 5.78 | 55.0/2.87 = 19.2 |
| (ii) | OxLDL × CRP/HDL | 0.85 × 3.38/49 = 0.0586 | 2.65 × 6.28/37.6 = 0.443 | 3.07 × 17.9/40.5 = 1.36 |
| | Advantage over control | — | 0.443/0.0586 = 7.56 | 1.36/0.0586 = 23.2 |
| (a) | OxLDL | 0.85 | 2.65 | 3.07 |
| | Advantage over control | — | 2.65/0.85 = 3.11 | 3.07/0.85 = 3.61 |
| (b) | CRP | 3.38 | 6.28 | 17.9 |
| | Advantage over control | — | 6.28/3.38 = 1.86 | 17.9/3.38 = 5.30 |
| (c) | HDL | 49 | 37.6 | 40.5 |
| | Advantage over control | — | 49/37.6 = 1.30 | 49/40.5 = 1.21 |
| (d) | CRP × Cholesterol | 3.38 × 180 = 608 | 6.28 × 180 = 1130 | 17.9 × 175 = 3133 |
| | Advantage over control | — | 1130/608 = 1.86 | 3133/608 = 5.15 |
| (e) | CRP × Cholesterol/HDL | 3.38 × 180/49 = 12.4 | 6.28 × 180/37.6 = 30.1 | 17.9 × 175/40.5 = 77.3 |
| | Advantage over control | — | 30.1/12.4 = 2.43 | 77.3/12.4 = 6.23 |

For purposes of evidencing the advantages of this invention, the assumption can be made that those in the control group are true negatives (this is believed to be a conservative assumption because some of the control individuals, all of whom are asymptomatic, may in fact have coronary artery disease). As will be understood by one skilled in the art, broadly speaking, for two given distributions (e.g., one distribution or curve of negatives and the other of positives), if the means of two distributions are farther apart (all else being equal), there will tend to be less overlap of the two distributions.

With the method of this invention, the OxLDL value multiplied by the CRP value for the Control sub-population is 2.87 (ignoring the units) and for the Stable (Chronic) Angina sub-population is 16.6 (again ignoring the units). Thus, the mean for the Stable (Chronic) Angina sub-population is 5.78 times the mean for the Control sub-population (16.6 divided by 2.87), which indicates a good separation between means and, therefore, less overlap. The mean for the Acute Coronary Syndromes sub-population is 19.2 times the mean for the Control sub-population (55.0 divided by 2.87), which indicates an even better separation between means and, therefore, still less overlap.

If instead of using OxLDL (an atherogenic protein) in combination with C-reactive protein, Total Cholesterol (a non-atherogenic protein) is used in combination with C-reactive protein, the Total Cholesterol value multiplied by the CRP value for the Control sub-population is 608 (ignoring the units) and for the Stable (Chronic) Angina sub-population is 1130 (again ignoring the units). The mean for the Stable (Chronic) Angina sub-population is only 1.86 times the mean for the Control sub-population (1130 divided by 608), which results in greater overlap of the distributions. Similarly, the mean for the Acute Coronary Syndromes sub-population is 5.15 times the mean for the Control sub-population (3133 divided by 608), but far less than for a combination within the invention, namely, using OxLDL and CRP together, for which the ratio was 19.2 (see preceding paragraph). (If MDA-modified LDL (instead of OxLDL) is used in combination with C-reactive protein or with C-reactive protein and HDL, broadly speaking, the separations are not as great as when OxLDL is used but are still better than when Total Cholesterol is used in combination with C-reactive protein or with C-reactive protein and HDL.)

Another way of quantifying the advantages of the present invention is to arithmetically compare the two levels of separation for Stable (Chronic) Angina (by dividing 5.78 (achieved with the invention) by 1.86 (resulting from not using the invention)) and for Acute Coronary Syndromes (by dividing 19.2 (achieved with the invention) by 5.15 (resulting from not using the invention)), which in each case equals more than 3. This indicates that use of atherogenic protein (OxLDL) plus acute phase reactant (C-reactive protein) (within the present invention) provides a separation of distribution means more than three times as great as the separation of distribution means achieved when using a non-atherogenic protein (Total Cholesterol) plus (C-reactive protein) (a combination outside the present invention).

In the same way, if an anti-atherogenic protein (e.g., HDL) is also used with the atherogenic protein and acute phase reactant (within the present invention), the mean for the Stable (Chronic) Angina sub-population is 7.56 times the mean for the Control group (0.443 divided by 0.0586). For the Acute Coronary Syndromes sub-population, the ratio is 23.2 (1.36 divided by 0.0586), i.e., the mean of the Acute Coronary Syndromes sub-population is over 23 times the mean of the Control sub-population when using the present invention with atherogenic protein, acute phase reactant, and anti-atherogenic protein. Accordingly, the separation of populations when using the invention (use of atherogenic protein (OxLDL), acute phase reactant (C-reactive protein), and anti-atherogenic protein (HDL)) as compared to when not using the invention (use of Total Cholesterol, C-reactive protein, and HDL) is again more than three times as great (for the Stable (Chronic) Angina sub-population 7.56 divided by 2.43 is more than 3 and for the Acute Coronary Syndromes sub-population 23.2 divided by 6.23 is also more than 3).

The separations achieved using the invention are far better than when any of OxLDL, C-reactive protein, or HDL is used alone. Considering first the Stable (Chronic) Angina sub-population, OxLDL alone results in a mean for the Stable (Chronic) Angina sub-population 3.11 times the mean of the Control sub-population, C-reactive protein alone results in a mean for the Stable (Chronic) Angina sub-population of 1.86 times the mean of the Control sub-population, and HDL alone results in a mean for the Stable (Chronic) Angina sub-population of 1.30 times the mean of the Control sub-population (each far less than the 5.78 for the invention when OxLDL and C-reactive protein are used together and each far less than the 7.56 for the invention when OxLDL, C-reactive protein, and HDL are used together).

With respect to the Acute Coronary Syndromes sub-population, OxLDL alone results in a mean for the Acute Coronary Syndromes sub-population 3.61 times the mean of the Control sub-population, C-reactive protein alone results in a mean for the Stable (Chronic) Angina sub-population 5.30 times the mean of the Control sub-population, and HDL alone results in a mean for the Stable (Chronic) Angina sub-population of 1.21 times the mean of the Control sub-population (each far less than the 19.2 for the invention when OxLDL and C-reactive protein are used together and each far less than the 23.2 for the invention when OxLDL, C-reactive protein, and HDL are used together).

In summary, the separation between the negatives (those in the Control sub-population) and the positives (those in either the Stable (Chronic) Angina or Acute Coronary Syndrome sub-population) is significantly greater when using the method of the invention (atherogenic protein plus acute phase reactant with or without anti-atherogenic protein) than when not using the invention (i.e., using Total Cholesterol plus C-reactive protein, or Total Cholesterol plus C-reactive protein plus HDL, or each of the three substances alone). These significant increases in separation are believed to sufficiently indicate the increased separation when using the method of the invention throughout the entire range of the disease, including distinguishing those with asymptomatic coronary artery disease from those without coronary artery disease.

It is believed that asymptomatic individuals with coronary artery disease would on average have values for OxLDL and C-reactive protein in combination not significantly lower than the values for individuals with stable angina. Thus, a single cut-point for using OxLDL and C-reactive protein together for detecting asymptomatic individuals with coronary artery disease could be established at a value of, for example, 12 (the Control sub-population has a mean of 2.87, the CAD-positive sub-populations (Stable (Chronic) Angina and Acute Coronary Syndromes) have means of 16.6 and 55, respectively, and the standard deviations for OxLDL and C-reactive protein individually are shown in Table III, above).

Using OxLDL and C-reactive protein together is the second combination shown in Table I, above. The value obtained from their use together is referred to herein as the third value related to both the level of the atherogenic protein and the level of the acute phase reactant, and the cut-point to which the third value is compared is the third cut-point and is related to both the atherogenic protein and the acute phase reactant (see Table I, above).

Use of OxLDL and C-reactive protein individually would be the first combination shown in Table I, above. The values obtained from their individual levels are the first value (related to the level of the atherogenic protein) and the second value (related to the level of the acute phase reactant), and the two cut-points are the first cut-point (related to the atherogenic protein) and the second cut-point (related to the acute phase reactant). The first cut-point (for the OxLDL) could be 1.4 and the second cut-point (for the C-reactive protein) could be 5.2 (see Table III, rows for OxLDL and C-reactive protein, which show means and standard deviations).

Variations and modifications will be apparent to those skilled in the art, and the claims are intended to cover all variations and modifications that fall within the true spirit and scope of the invention.

As noted above, substances besides the three specified (i.e., atherogenic protein, acute phase reactant, and optional anti-atherogenic protein) can be used in addition to those three, either alone or in combination with those three, and the claims are intended to cover such use provided that at least atherogenic protein and acute phase reactant are used. Thus, the use of such other substances does not prevent a value based at least in part on one of those three (i.e., atherogenic protein, acute phase reactant, and optional anti-atherogenic protein) from being “related to” that one or more of the three. For example, a value based on the level of an atherogenic protein as well as on the level of a fourth substance (i.e., a substance that is not atherogenic protein, acute phase reactant, or anti-atherogenic protein) is still related to the level of the atherogenic protein (e.g., the level of the atherogenic protein multiplied by the level of the fourth substance is related to the level of the atherogenic protein).

In performing the calculations to determine the values shown in Table IV, row (i), the level of OxLDL (the atherogenic protein) was multiplied by the level of C-reactive protein (the acute phase reactant). Similarly, in performing the calculations to determine the values shown in Table IV, row (ii), the level of OxLDL (the atherogenic protein) was multiplied by the level of C-reactive protein (the acute phase reactant) and divided by the level of HDL (anti-atherogenic protein).

In some cases (e.g., with a different atherogenic protein and/or a different C-reactive protein and/or a different anti-atherogenic protein, or even with OxLDL and C-reactive protein and HDL) it may be desirable to use weighting factors (e.g., 0.6 OxLDL level multiplied by 0.5 C-reactive protein level). As indicated above, any qualitative or quantitative relationship may be employed when two or more of the three categories of substances (atherogenic protein, acute phase reactant, and optional anti-atherogenic protein) are used together.

1. A method of making an assessment of the likelihood that a human patient who is asymptomatic for coronary artery disease has the disease, the method comprising the steps:
(a) obtaining the level of an atherogenic protein in a sample from the patient, obtaining the level of an acute phase reactant in a sample from the patient, and optionally obtaining the level of an anti-atherogenic protein in a sample from the patient;
(b) obtaining at least one of:
(i) a first cut-point related to the atherogenic protein and a second cut-point related to the acute phase reactant,
(ii) a third cut-point related to the atherogenic protein and the acute phase reactant,
(iii) a fourth cut-point related to the atherogenic protein and the acute phase reactant and a fifth cut-point related to the anti-atherogenic protein,
(iv) a sixth cut-point related to the atherogenic protein and a seventh cut-point related to the acute phase reactant and the anti-atherogenic protein,
(v) an eighth cut-point related to the atherogenic protein and the anti-atherogenic protein and a ninth cut-point related to the acute phase reactant,
(vi) a tenth cut-point related to the atherogenic protein, the acute phase reactant, and the anti-atherogenic protein,
(vii) an eleventh cut-point related to the atherogenic protein, a twelfth cut-point related to the acute phase reactant, and a thirteenth cut-point related to the anti-atherogenic protein; and
(c) assessing whether the patient is likely to have asymptomatic coronary artery disease based on at least one of the following:
(i) a comparison to the first cut-point of a first value related to the level of the atherogenic protein and a comparison to the second cut-point of a second value related to the level of the acute phase reactant,
(ii) a comparison to the third cut-point of a third value related to the levels of the atherogenic protein and acute phase reactant,
(iii) a comparison to the fourth cut-point of a fourth value related to the levels of the atherogenic protein and acute phase reactant and a comparison to the fifth cut-point of a fifth value related to the level of the anti-atherogenic protein,
(iv) a comparison to the sixth cut-point of a sixth value related to the level of the atherogenic protein and a comparison to the seventh cut-point of a seventh value related to the levels of the acute phase reactant and anti-atherogenic protein,
(v) a comparison to the eighth cut-point of an eighth value related to the levels of the atherogenic protein and anti-atherogenic protein and a comparison to the ninth cut-point of a ninth value related to the level of the acute phase reactant,
(vi) a comparison to the tenth cut-point of a tenth value related to the levels of the atherogenic protein, acute phase reactant, and anti-atherogenic protein, and
(vii) a comparison to the eleventh cut-point of an eleventh value related to the level of the atherogenic protein, a comparison to the twelfth cut-point of a twelfth value related to the level of the acute phase reactant, and a comparison to the thirteenth cut-point of a thirteenth value related to the level of the anti-atherogenic protein.

2. The method of claim 1 wherein the atherogenic protein comprises OxLDL (oxidized low density lipoprotein).

3. The method of claim 2 wherein the OxLDL contains at least 60 substituted lysine residues per apo B-100 (apolipoprotein B-100) moiety.

4. The method of claim 1 wherein the acute phase reactant comprises a positive acute phase reactant or a negative acute phase reactant.

5. The method of claim 4 wherein the positive acute phase reactant is selected from the group consisting of C-reactive protein, serum amyloid A, von Willebrand factor, ferritin, and fibrinogen and the negative acute phase reactant is selected from the group consisting of albumin, apo A-I (apolipoprotein A-I), apo A-II (apolipoprotein A-II), and HDL (high density lipoprotein).

6. The method of claim 1 wherein the anti-atherogenic protein comprises HDL.

7. The method of claim 1 wherein the atherogenic protein comprises OxLDL and the acute phase reactant is selected from the group consisting of C-reactive protein and fibrinogen.

8. The method of claim 7 wherein the anti-atherogenic protein comprises HDL.

9. The method of claim 1 wherein step (a) uses an immunological assay to obtain the level of atherogenic protein.

10. The method of claim 9 wherein the immunological assay uses one or more monoclonal antibodies each having an affinity for the atherogenic protein of at least about 5×10⁸ M⁻¹.

11. The method of claim 10 wherein the immunological assay uses at least one of the following monoclonal antibodies to obtain the level of atherogenic protein: mAb-4E6 produced by hybridoma Hyb4E6 deposited with the BCCM (Belgian Coordinated Collections of Microorganisms) under deposit accession number LMBP 1660 CB, mAb-1H 11 produced by hybridoma Hyb1H11 deposited with the BCCM under deposit accession number LMBP 1659 CB, and mAb-8A2 produced by hybridoma Hyb8A2 deposited with the BCCM under deposit accession number LMBP 1661 CB.

12. The method of claim 1 wherein the atherogenic protein comprises an atherogenic low density lipoprotein and step (a) is conducted using an immunological assay.

13. The method of claim 1 wherein the atherogenic protein comprises OxLDL and an immunological assay is used to obtain the level of atherogenic protein.

14. The method of claim 13 wherein the immunological assay uses one or more monoclonal antibodies each having an affinity for the atherogenic protein of at least about 5×10⁸ M⁻¹.

15. The method of claim 13 wherein in step (a) the level of anti-atherogenic protein is obtained and the anti-atherogenic protein comprises HDL.

16. The method of claim 13 wherein the acute phase reactant is selected from the group consisting of C-reactive protein and fibrinogen.

17. The method of claim 16 wherein in step (a) the level of anti-atherogenic protein is obtained and the anti-atherogenic protein comprises HDL.

18. The method of claim 13 wherein the OxLDL contains at least 60 substituted lysine residues per apo B-100 moiety.

19. The method of claim 18 wherein in step (a) the level of anti-atherogenic protein is obtained and the anti-atherogenic protein comprises HDL.

20. The method of claim 19 wherein the acute phase reactant is selected from the group consisting of C-reactive protein and fibrinogen.

21. The method of claim 12 wherein the immunological assay uses at least one of the following monoclonal antibodies to obtain the level of atherogenic protein: mAb-4E6 produced by hybridoma Hyb4E6 deposited with the BCCM under deposit accession number LMBP 1660 CB, mAb-1H11 produced by hybridoma Hyb1H11 deposited with the BCCM under deposit accession number LMBP 1659 CB, and mAb-8A2 produced by hybridoma Hyb8A2 deposited with the BCCM under deposit accession number LMBP 1661 CB.

22. A method of facilitating the assessment by a medical professional of the likelihood that a human patient who is asymptomatic for coronary artery disease has the disease, the method comprising the steps:
(a) obtaining the level of an atherogenic protein in a sample from the patient, obtaining the level of an acute phase reactant in a sample from the patient, and optionally obtaining the level of an anti-atherogenic protein in a sample from the patient;
(b) obtaining at least one of:
(i) a first cut-point related to the atherogenic protein and a second cut-point related to the acute phase reactant,
(ii) a third cut-point related to the atherogenic protein and the acute phase reactant,
(iii) a fourth cut-point related to the atherogenic protein and the acute phase reactant and a fifth cut-point related to the anti-atherogenic protein,
(iv) a sixth cut-point related to the atherogenic protein and a seventh cut-point related to the acute phase reactant and the anti-atherogenic protein,
(v) an eighth cut-point related to the atherogenic protein and the anti-atherogenic protein and a ninth cut-point related to the acute phase reactant,
(vi) a tenth cut-point related to the atherogenic protein, the acute phase reactant, and the anti-atherogenic protein, and
(vii) an eleventh cut-point related to the atherogenic protein, a twelfth cut-point related to the acute phase reactant, and a thirteenth cut-point related to the anti-atherogenic protein;
(c) providing to the medical professional at least one of:
(i) a first value related to the level of the atherogenic protein and a second value related to the level of the acute phase reactant,
(ii) a third value related to the levels of the atherogenic protein and acute phase reactant,
(iii) a fourth value related to the levels of the atherogenic protein and acute phase reactant and a fifth value related to the level of the anti-atherogenic protein,
(iv) a sixth value related to the level of the atherogenic protein and a seventh value related to the levels of the acute phase reactant and anti-atherogenic protein,
(v) an eighth value related to the levels of the atherogenic protein and anti-atherogenic protein and a ninth value related to the level of the acute phase reactant, and
(vi) a tenth value related to the levels of the atherogenic protein, acute phase reactant, and anti-atherogenic protein,
(vii) an eleventh value related to the level of the atherogenic protein, a twelfth value related to the level of the acute phase reactant, and a thirteenth value related to the level of the anti-atherogenic protein; and
(d) providing to the medical professional the appropriate one or more of the cut-points to permit the medical professional to assess whether the patient is likely to have asymptomatic coronary artery disease based on at least one of the following:
(i) a comparison to the first cut-point of the first value and a comparison to the second cut-point of the second value,
(ii) a comparison to the third cut-point of the third value,
(iii) a comparison to the fourth cut-point of the fourth value and a comparison to the fifth cut-point of the fifth value,
(iv) a comparison to the sixth cut-point of the sixth value and a comparison to the seventh cut-point of the seventh value,
(v) a comparison to the eighth cut-point of the eighth value and a comparison to the ninth cut-point of the ninth value,
(vi) a comparison to the tenth cut-point of the tenth value, and
(vii) a comparison to the eleventh cut-point of the eleventh value, a comparison to the twelfth cut-point of the twelfth value, and a comparison to the thirteenth cut-point of the thirteenth value.

23. The method of claim 22 wherein the atherogenic protein comprises OxLDL.

24. The method of claim 23 wherein the OxLDL contains at least 60 substituted lysine residues per apo B-100 moiety.

25. The method of claim 22 wherein the acute phase reactant comprises a positive acute phase reactant or a negative acute phase reactant.

26. The method of claim 25 wherein the positive acute phase reactant is selected from the group consisting of C-reactive protein, serum amyloid A, von Willebrand factor, ferritin, and fibrinogen and the negative acute phase reactant is selected from the group consisting of albumin, apo A-I, apo A-II, and HDL.

27. The method of claim 22 wherein the anti-atherogenic protein comprises HDL.

28. The method of claim 22 wherein the atherogenic protein comprises OxLDL and the acute phase reactant is selected from the group consisting of C-reactive protein and fibrinogen.

29. The method of claim 28 wherein the anti-atherogenic protein is HDL.

30. The method of claim 22 wherein step (a) uses an immunological assay to obtain the level of atherogenic protein.

31. The method of claim 30 wherein the immunological assay uses one or more monoclonal antibodies each having an affinity for the atherogenic protein of at least about 5×10⁸ M⁻¹.

32. The method of claim 30 wherein the immunological assay uses at least one of the following monoclonal antibodies to obtain the level of atherogenic protein: mAb-4E6 produced by hybridoma Hyb4E6 deposited with the BCCM under deposit accession number LMBP 1660 CB, mAb-1H11 produced by hybridoma Hyb1H11 deposited with the BCCM under deposit accession number LMBP 1659 CB, and mAb-8A2 produced by hybridoma Hyb8A2 deposited with the BCCM under deposit accession number LMBP 1661 CB.

33. The method of claim 22 wherein the atherogenic protein comprises an atherogenic low density lipoprotein and step (a) is conducted using an immunological assay.

34. The method of claim 22 wherein the atherogenic protein comprises OxLDL and an immunological assay is used to obtain the level of atherogenic protein.

35. The method of claim 34 wherein the immunological assay uses one or more monoclonal antibodies each having an affinity for the atherogenic protein of at least about 5×10⁸ M⁻¹.

36. The method of claim 34 wherein in step (a) the level of anti-atherogenic protein is obtained and the anti-atherogenic protein comprises HDL.

37. The method of claim 34 wherein the acute phase reactant is selected from the group consisting of C-reactive protein and fibrinogen.

38. The method of claim 37 wherein in step (a) the level of anti-atherogenic protein is obtained and the anti-atherogenic protein comprises HDL.

39. The method of claim 34 wherein the OxLDL contains at least 60 substituted lysine residues per apo B-100 moiety.

40. The method of claim 39 wherein in step (a) the level of anti-atherogenic protein is obtained and the anti-atherogenic protein comprises HDL.

41. The method of claim 40 wherein the acute phase reactant is selected from the group consisting of C-reactive protein and fibrinogen.

42. The method of claim 33 wherein the immunological assay uses at least one of the following monoclonal antibodies to obtain the level of atherogenic protein: mAb-4E6 produced by hybridoma Hyb4E6 deposited with the BCCM under deposit accession number LMBP 1660 CB, mAb-1H11 produced by hybridoma Hyb1H11 deposited with the BCCM under deposit accession number LMBP 1659 CB, and mAb-8A2 produced by hybridoma Hyb8A2 deposited with the BCCM under deposit accession number LMBP 1661 CB.
